(19) United States
(12) Patent Application Publication
(10) Pub. No.: US 2015/0140449 A1
(43) Pub. Date: May 21, 2015

(54) COMPOUND, NONAQUEOUS ELECTROLYTE, AND POWER STORAGE DEVICE

(71) Applicant: SEMICONDUCTOR ENERGY LABORATORY CO., LTD., Atsugi-shi (JP)

(72) Inventors: Jun ISHIKAWA, Atsugi (JP); Satoshi SEO, Sagamihara (JP); Rie YOKOI, Atsugi (JP); Hiroshi KADOMA, Sagamihara (JP); Tomoya HIROSE, Atsugi (JP)

(21) Appl. No.: 14/537,975

(22) Filed: Nov. 11, 2014

(30) Foreign Application Priority Data
Nov. 15, 2013 (JP) 2013-237147
Nov. 15, 2013 (JP) 2013-237158
Jul. 23, 2014 (JP) 2014-149489

(51) Int. Cl. H01M 10/0569; C07D 233/60; H01M 10/0525

(52) U.S. Cl. CPC H01M 10/0569; H01M 10/0525; C07D 233/60; H01M 2300/0034; H01M 2220/20; H01M 2220/30
USPC 429/336; 548/341.1

(57) ABSTRACT

Provided are a nonaqueous solvent containing a compound with high conductivity and low viscosity and a high-performance power storage device using the nonaqueous solvent. The power storage device includes an ionic liquid. The ionic liquid contains an anion and a cation having a five-membered heteroaromatic ring having one or more substituents. At least one of the substituents is a straight chain formed of four or more atoms and includes one or more of C, O, Si, N, S, and P.

### BACKGROUND OF THE INVENTION

1. Field of the Invention

One embodiment of the present invention relates to a compound, a nonaqueous electrolyte including the compound, and a power storage device including the nonaqueous electrolyte.

Note that one embodiment of the present invention is not limited to the above technical field. The technical field of one embodiment of the invention disclosed in this specification and the like relates to an object, a method, or a manufacturing method. In addition, one embodiment of the present invention relates to a process, a machine, manufacture, or a composition of matter. Specifically, examples of the technical field of one embodiment of the present invention disclosed in this specification include a semiconductor device, a display device, a light-emitting device, a power storage device, a storage device, a method for driving any of them, and a method for manufacturing any of them.

Note that the power storage device indicates all elements and devices which have a function of storing power.

2. Description of the Related Art

In recent years, a variety of power storage devices such as lithium-ion secondary batteries, lithium-ion capacitors, and air batteries have been actively developed. In particular, demand for lithium-ion secondary batteries with high output and high energy density has rapidly grown with the development of the semiconductor industry, for the uses of electronic devices, for example, portable information terminals such as mobile phones, smartphones, and laptop computers, portable music players, and digital cameras; medical equipment; and next-generation clean energy vehicles such as hybrid electric vehicles (HEVs), electric vehicles (EVs), and plug-in hybrid electric vehicles (PHEVs). The lithium-ion secondary batteries are essential for today's information society as rechargeable energy supply sources.

As described above, lithium-ion secondary batteries have been used for a variety of purposes in various fields. Properties necessary for such lithium-ion secondary batteries are high energy density, excellent cycle characteristics, safety in a variety of operation environments, and the like.

Many of the widely used lithium-ion secondary batteries include a nonaqueous electrolyte (also referred to as a nonaqueous electrolyte solution) including a nonaqueous solvent and a lithium salt containing lithium ions. As the nonaqueous electrolyte, an organic solvent which has high dielectric constant and excellent ionic conductivity, such as ethylene carbonate, is often used.

However, the above-described organic solvent has volatility and a low flash point. For this reason, when the organic solvent is used in a lithium-ion secondary battery, the internal temperature of the lithium-ion secondary battery might rise because of short-circuit, overcharging, or the like, and the lithium-ion secondary battery would explode or catch fire.

In view of the above, the use of an ionic liquid (also referred to as a room temperature molten salt) which has non-flammability and non-volatility as a nonaqueous solvent for a nonaqueous electrolyte of a lithium-ion secondary battery has been proposed. Examples of such an ionic liquid are an ionic liquid containing an ethylmethylimidazolium (EMI) cation, an ionic liquid containing an N-methyl-N-propylpyrrolidinium (P13) cation, and an ionic liquid containing an N-methyl-N-propylpiperidinium (PP13) cation (see Patent Document 1).

Improvements are made to an anion component and a cation component of an ionic liquid to provide a lithium-ion secondary battery which uses an ionic liquid with low viscosity, a low melting point, and high conductivity (see Patent Document 2).

### REFERENCE

### Patent Document

[Patent Document 1] Japanese Published Patent Application No. 2003-331918
[Patent Document 2] PCT International Publication No. WO2005063773

### SUMMARY OF THE INVENTION

As a solvent for a nonaqueous electrolyte of a lithium-ion secondary battery, nonaqueous solvents, typified by an ionic liquid, have been developed. However, there is room for improvement in various points such as viscosity, a melting point, conductivity, and cost. A more excellent nonaqueous solvent is desired to be developed.

For example, in the case where an ionic liquid containing a cation of an aliphatic compound is used as a nonaqueous solvent, the nonaqueous solvent has low ionic conductivity (e.g., lithium ion conductivity) because the ionic liquid has high viscosity. Furthermore, in the case of a lithium-ion secondary battery using the ionic liquid, resistance of the ionic liquid (specifically, an electrolyte including the ionic liquid) is increased in a low temperature environment (particularly at 0° C. or lower) and thus the lithium-ion secondary battery does not operate properly.

Moreover, an ionic liquid containing an imidazolium cation has poor cycle characteristics at high temperature in some cases. This is probably due to reductive decomposition derived by a low reduction potential of the imidazolium cation. Thus, it might be difficult to drastically change the reduction potential of the imidazolium cation having an imidazole ring.

In view of the above, an object of one embodiment of the present invention is to provide a compound used for an ionic liquid that makes a nonaqueous solvent including the ionic liquid containing the compound have at least one of the following characteristics: high lithium conductivity in a low temperature environment, high heat resistance, a wide usable temperature range, a low freezing point (melting point), low viscosity, and the like. Another object of one embodiment of the present invention is to provide a power storage device including a nonaqueous electrolyte including the ionic liquid. The nonaqueous electrolyte has at least one of the following characteristics: high lithium conductivity, high lithium conductivity in a low temperature environment, high heat resistance, a wide temperature range, a low freezing point (melting point), low viscosity, and the like. Another object of one embodiment of the present invention is to provide a compound that improves cycle characteristics of a power storage device at high temperature. Another object of one embodiment of the present invention is to provide a compound with a high reduction potential. Another object of one embodiment of the present invention is to provide a novel compound.

Another object of one embodiment of the present invention is to provide a nonaqueous solvent containing a compound which allows fabrication of a high-performance power storage device. Another object of one embodiment of the present invention is to provide a high-performance power storage device. Another object of one embodiment of the present invention is to provide a power storage device with a high degree of safety. Another object of one embodiment of the present invention is to provide a novel power storage device.

Note that the description of these objects does not impede the existence of other objects. In one embodiment of the present invention, there is no need to achieve all the above objects. Other objects will be apparent from and can be derived from the description of the specification, the drawings, the claims, and the like.

For one embodiment of the present invention, an ionic liquid with low viscosity containing an imidazolium cation is used. One embodiment of the present invention is a compound including an anion and a cation represented by General Formula (G1). The anion is any one of a monovalent amide anion, a monovalent methide anion, a fluorosulfonate anion (SO₃F⁻), a perfluoroalkylsulfonate anion, a tetrafluoroborate anion (BF₄⁻), a perfluoroalkylborate anion, a hexafluorophosphate anion (PF₆⁻), and a perfluoroalkylphosphate anion.

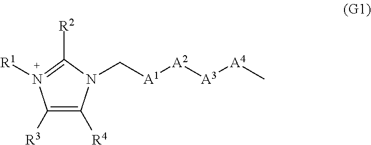

In General Formula (G1), R¹ represents an alkyl group having 1 to 4 carbon atoms, R² to R⁴ each independently represent a hydrogen atom or an alkyl group having 1 to 4 carbon atoms, A¹ to A⁴ each independently represent a methylene group or an oxygen atom, and at least one of A¹ to A⁴ represents an oxygen atom.

Another embodiment of the present invention is a compound including an anion and a cation represented by General Formula (G1). The anion is a bis(fluorosulfonyl)amide anion.

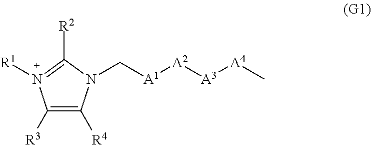

In General Formula (G1), R¹ represents an alkyl group having 1 to 4 carbon atoms, R² to R⁴ each independently represent a hydrogen atom or an alkyl group having 1 to 4 carbon atoms, A¹ to A⁴ each independently represent a methylene group or an oxygen atom, and at least one of A¹ to A⁴ represents an oxygen atom.

Another embodiment of the present invention is a compound including an anion and a cation represented by General Formula (G1). The anion is a bis(fluorosulfonyl)amide anion.

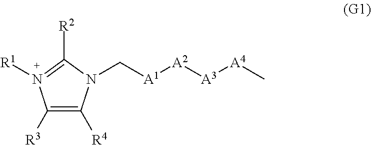

In General Formula (G1), R¹ represents an alkyl group having 1 to 4 carbon atoms, R² to R⁴ each independently represent a hydrogen atom or a methyl group, A¹ to A⁴ each independently represent a methylene group or an oxygen atom, and at least one of A¹ to A⁴ represents an oxygen atom.

Another embodiment of the present invention is a compound including a cation represented by General Formula (G2) and a monovalent anion.

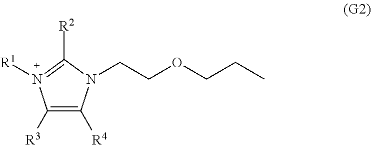

In General Formula (G2), R¹ represents an alkyl group having 1 to 4 carbon atoms, R² to R⁴ each independently represent a hydrogen atom or an alkyl group having 1 to 4 carbon atoms.

Another embodiment of the present invention is a compound including an anion and a cation represented by General Formula (G2). The anion is any one of a monovalent amide anion, a monovalent methide anion, a fluorosulfonate anion (SO₃F⁻), a perfluoroalkylsulfonate anion, a tetrafluoroborate anion (BF₄⁻), a perfluoroalkylborate anion, a hexafluorophosphate anion (PF₆⁻), and a perfluoroalkylphosphate anion.

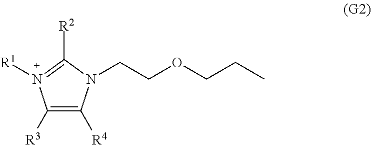

In General Formula (G2), R¹ represents an alkyl group having 1 to 4 carbon atoms, R² to R⁴ each independently represent a hydrogen atom or an alkyl group having 1 to 4 carbon atoms.

Another embodiment of the present invention is a compound including an anion and a cation represented by General Formula (G3). The anion is any one of a monovalent amide anion, a monovalent methide anion, a fluorosulfonate anion (SO₃F⁻), a perfluoroalkylsulfonate anion, a tetrafluoroborate anion (BF₄⁻), a perfluoroalkylborate anion, a hexafluorophosphate anion (PF₆⁻), and a perfluoroalkylphosphate anion.

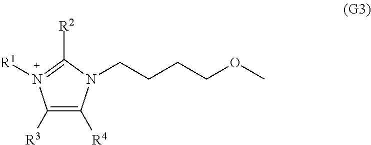

In General Formula (G3), R¹ represents an alkyl group having 1 to 4 carbon atoms, R² to R⁴ each independently represent a hydrogen atom or an alkyl group having 1 to 4 carbon atoms.

Another embodiment of the present invention is a nonaqueous electrolyte including an alkali metal salt and a nonaqueous solvent. The nonaqueous solvent includes any one of the above compounds.

Another embodiment of the present invention is a power storage device including an ionic liquid. The ionic liquid contains an anion and a cation having a five-membered heteroaromatic ring. The five-membered heteroaromatic ring has one or more substituents. At least one of the substituents is a straight chain formed of four or more atoms and includes one or more of C, O, Si, N, S, and P.

Another embodiment of the present invention is a power storage device including an ionic liquid. The ionic liquid contains an anion and a cation having a monocyclic five-membered heteroaromatic ring. The monocyclic five-membered heteroaromatic ring has one or more substituents. At least one of the substituents is a straight chain formed of four or more atoms and includes one or more of C, O, Si, N, S, and P.

In the above structure, at least one heteroatom in the five-membered heteroaromatic ring preferably has the straight chain.

Another embodiment of the present invention is a power storage device including an ionic liquid. The ionic liquid contains an anion and a cation having a five-membered heteroaromatic ring. The five-membered heteroaromatic ring has one or more substituents. The five-membered heteroaromatic ring includes at least one nitrogen atom. At least one of the substituents is a straight chain formed of four or more atoms and includes one or more of C, O, Si, N, S, and P.

Another embodiment of the present invention is a power storage device including an ionic liquid. The ionic liquid contains an anion and a cation having a monocyclic five-membered heteroaromatic ring. The monocyclic five-membered heteroaromatic ring has one or more substituents. At least one of the substituents is a straight chain formed of four or more atoms and includes one or more of C, O, Si, N, S, and P.

In the above structure, at least one nitrogen atom in the five-membered heteroaromatic ring preferably has the straight chain.

In the above structure, the cation having the monocyclic five-membered heteroaromatic ring is an imidazolium cation.

Another embodiment of the present invention is a power storage device including a nonaqueous electrolyte containing an ionic liquid and an alkali metal salt. The ionic liquid contains an anion and a cation having a five-membered heteroaromatic ring. The five-membered heteroaromatic ring has one or more substituents. At least one of the substituents is a straight chain formed of four or more atoms and includes one or more of C, O, Si, N, S, and P.

Another embodiment of the present invention is a power storage device including a nonaqueous electrolyte containing an ionic liquid and an alkali metal salt. The ionic liquid contains an anion and a cation having a monocyclic five-membered heteroaromatic ring. The monocyclic five-membered heteroaromatic ring has one or more substituents. At least one of the substituents is a straight chain formed of four or more atoms and includes one or more of C, O, Si, N, S, and P.

In the above structure, at least one heteroatom in the five-membered heteroaromatic ring preferably has the straight chain.

Another embodiment of the present invention is a power storage device including a nonaqueous electrolyte containing an ionic liquid and an alkali metal salt. The ionic liquid contains an anion and a cation having a five-membered heteroaromatic ring. The five-membered heteroaromatic ring has one or more substituents. The five-membered heteroaromatic ring includes at least one nitrogen atom. At least one of the substituents is a straight chain formed of four or more atoms and includes one or more of C, O, Si, N, S, and P.

Another embodiment of the present invention is a power storage device including a nonaqueous electrolyte containing an ionic liquid and an alkali metal salt. The ionic liquid contains an anion and a cation having a monocyclic five-membered heteroaromatic ring. The monocyclic five-membered heteroaromatic ring has one or more substituents. At least one of the substituents is a straight chain formed of four or more atoms and includes one or more of C, O, Si, N, S, and P.

In the above structure, at least one nitrogen atom in the five-membered heteroaromatic ring preferably has the straight chain.

In the above structure, the cation having the monocyclic five-membered heteroaromatic ring is an imidazolium cation.

In the above structure, the alkali metal salt is preferably a lithium salt.

One embodiment of the present invention can provide a compound for an ionic liquid with which a high-performance power storage device can be fabricated. Furthermore, a high-performance power storage device can be provided. One embodiment of the present invention can provide a power storage device with a high degree of safety. One embodiment of the present invention can provide a novel compound. One embodiment of the present invention can provide a novel power storage device. Note that the description of these effects does not disturb the existence of other effects. One embodiment of the present invention does not necessarily achieve all the objects listed above. Other effects will be apparent from and can be derived from the description of the specification, the drawings, the claims, and the like.

### BRIEF DESCRIPTION OF THE DRAWINGS

FIGS. 1A to 1C illustrate a coin-type secondary battery of one embodiment of the present invention.

FIGS. 2A and 2B illustrate a cylindrical storage battery of one embodiment of the present invention.

FIGS. 3A and 3B illustrate a thin secondary battery of one embodiment of the present invention.

FIGS. 4A and 4B illustrate a thin secondary battery of one embodiment of the present invention.

FIGS. 5A to 5C illustrate thin secondary batteries of one embodiment of the present invention.

FIGS. 6A to 6C illustrate rectangular secondary batteries of one embodiment of the present invention.

FIGS. 7A and 7B illustrate a power storage device of one embodiment of the present invention.

FIGS. 8A1, 8A2, 8B1, and 8B2 illustrate a power storage device of one embodiment of the present invention.

FIGS. 9A and 9B illustrate power storage devices of one embodiment of the present invention.

FIGS. 10A to 10F illustrate electronic devices each including a flexible secondary battery of one embodiment of the present invention.

FIGS. 11A and 11B illustrate vehicles of one embodiment of the present invention.

FIG. 12 is a ¹H NMR chart of an intermediate of an ionic liquid of one embodiment of the present invention.

FIG. 13 is a ¹H NMR chart of an ionic liquid of one embodiment of the present invention.

FIG. 14 is a ¹H NMR chart of an intermediate of an ionic liquid of one embodiment of the present invention.

FIG. 15 is a ¹H NMR chart of an ionic liquid of one embodiment of the present invention.

FIG. 16 is a ¹H NMR chart of an ionic liquid of one embodiment of the present invention.

FIG. 17 is a ¹H NMR chart of an intermediate of an ionic liquid of one embodiment of the present invention.

FIG. 18 is a ¹H NMR chart of an ionic liquid of one embodiment of the present invention.

FIG. 19 is a ¹H NMR chart of an intermediate of an ionic liquid of one embodiment of the present invention.

FIG. 20 is a ¹H NMR chart of an ionic liquid of one embodiment of the present invention.

FIGS. 21A and 21B illustrate coin cell structures of Example.

FIGS. 22A and 22B show the measurement results of initial charge and discharge characteristics of samples of Example.

FIGS. 23A and 23B show the measurement results of initial charge and discharge characteristics of samples of Example.

FIGS. 24A and 24B show the measurement results of initial charge and discharge characteristics of samples of Example.

FIG. 25 shows the measurement results of initial charge and discharge characteristics of a sample of Example.

FIGS. 26A and 26B show the initial charge and discharge efficiencies and cycle characteristics of samples of Example.

FIG. 27 shows the measurement results of rate characteristics of samples of Example.

FIG. 28 shows the results of aging of samples of Example.

FIG. 29 shows the measurement results of cycle characteristics of samples of Example.

FIGS. 30A and 30B show the charge and discharge characteristics and the measurement results of rate characteristics of a sample of Example.

FIGS. 31A and 31B show the charge and discharge characteristics and the measurement results of rate characteristics of a sample of Example.

FIGS. 32A and 32B show the charge and discharge characteristics and the measurement results of temperature characteristics of a sample of Example.

FIGS. 33A and 33B show the charge and discharge characteristics and the measurement results of temperature characteristics of a sample of Example.

FIGS. 34A and 34B show results of differential scanning calorimetry measurement of samples of Example.

### DETAILED DESCRIPTION OF THE INVENTION

Embodiments of the present invention will be described below in detail with reference to the drawings. Note that the present invention is not limited to the following description. It will be readily understood by those skilled in the art that modes and details of the present invention can be changed in various ways without departing from the spirit and scope of the present invention. Therefore, the present invention should not be construed as being limited to the following description of the embodiments. In describing structures of the present invention with reference to the drawings, common reference numerals are used for the same portions in different drawings. The same hatching pattern is applied to similar parts, and the similar parts are not especially denoted by reference numerals in some cases. Note that the size, the layer thickness, or the region of each structure illustrated in the drawings might be exaggerated for the sake of clarity. Thus, the present invention is not necessarily limited to such scales illustrated in the drawings.

### Embodiment 1

In this embodiment, a nonaqueous solvent used in a power storage device of one embodiment of the present invention is described.

A nonaqueous solvent used for a power storage device of one embodiment of the present invention includes an ionic liquid. The ionic liquid contains an anion and a cation having a five-membered heteroaromatic ring having one or more substituents.

In the cation having a five-membered heteroaromatic ring in the ionic liquid, at least one of the substituents is a straight chain formed of four or more atoms and includes one or more of C, O, Si, N, S, and P. The straight chain may have a substituent (including a side chain). The substituent of the straight chain is, for example, an alkyl group or an alkoxy group.

Since at least one of the substituents has the straight chain, the cation in the ionic liquid has sterically bulky structure and thus side reactions (e.g., cation insertion into graphite and decomposition of the nonaqueous solvent during charging, and gas generation associated with the insertion and the decomposition) in a battery can be suppressed. However, the viscosity of the ionic liquid is likely to be enhanced as the number of carbon atoms in the straight chain is larger; therefore, it is preferable to determine the number of carbon atoms in the straight chain in accordance with desirable charge and discharge efficiency and desirable viscosity.

Examples of the cation having a five-membered heteroaromatic ring in the ionic liquid, are a benzimidazolium cation, a benzoxazolium cation, and a benzothiazolium cation. Examples of the cation having a monocyclic five-membered heteroaromatic ring include an oxazolium cation, a thiazolium cation, an isoxazolium cation, an isothiazolium cation, an imidazolium cation, and a pyrazolium cation. In view of the stability, viscosity, and ion conductivity of the compound and ease of synthesis, the cation having a monocyclic five-membered heteroaromatic ring is preferred. In particular, an imidazolium cation is preferred because it can make the viscosity low.

The anion included in the ionic liquid is a monovalent anion which forms the ionic liquid with the cation having a five-membered heteroaromatic ring. Examples of the anion include a monovalent amide anion, a monovalent methide anion, a fluorosulfonate anion (SO₃F⁻), a perfluoroalkylsulfonate anion, a tetrafluoroborate anion (BF₄⁻), a perfluoroalkylborate anion, a hexafluorophosphate anion (PF₆⁻), and a perfluoroalkylphosphate anion. An example of the monovalent amide anion is (CₙF₂ₙ₊₁SO₂)₂N⁻ (n=0 to 3). An example of the monovalent cyclic amide anion is (CF₂SO₂)₂N⁻. An example of the monovalent methide anion is (CₙF₂ₙ+1SO₂)₃C⁻ (n 0 to 3). An example of the monovalent cyclic methide anion is (CF₂SO₂)₂C⁻(CF₃SO₂). An example of the perfluoroalkylsulfonate anion is (CₘF₂ₘ₊₁SO₃)⁻ (m=0 to 4). An example of the perfluoroalkylborate anion, {BFₙ(CₘHₖF₂ₘ₊₊₁₋ₖ)₄₋ₙ}⁻ (n=0 to 3, m=1 to 4, and k=0 to 2m). An example of the perfluoroalkylphosphate anion is {PFₙ(CₘHₖF₂ₘ₊₁₋ₖ)₆₋ₙ}⁻ (n=0 to 5, m=1 to 4, and k=0 to 2m). Note that the anion is not limited thereto.

The ionic liquid which can be used in the nonaqueous solvent of the power storage device of one embodiment of the present invention can be represented by General Formula (G0), for example.

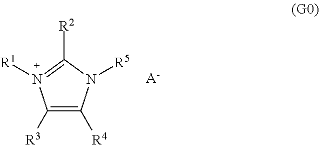

In General Formula (G0), R¹ represents an alkyl group having 1 to 4 carbon atoms; R² to R⁴ each independently represent a hydrogen atom or an alkyl group having 1 to 4 carbon atoms; R⁵ represents a straight chain formed of four or more atoms, and includes one or more of C, O, Si, N, S, and P; and A⁻ represents any one of a monovalent amide anion, a monovalent methide anion, a fluorosulfonate anion, a perfluoroalkylsulfonate anion, a tetrafluoroborate anion, a perfluoroalkylborate anion, a hexafluorophosphate anion, and a fluoroalkylphosphate anion.

The straight chain represented by R⁵ may have a substituent. Examples of the substituent include an alkyl group and an alkoxy group.

Note that in General Formula (G0), R⁵ represents the straight chain formed of four or more atoms and includes one or more of C, O, Si, N, S, and P, but the position of the straight chain is not limited to R⁵; R² or R³ may represent the straight chain. In addition, two or more of R¹ to R⁵ (e.g., R¹ and R⁵, R² and R⁵, R² and R³, or R′, R², and R⁵) may represent the straight chains.

The alkyl group in the cation represented by General Formula (G0) may be either a straight-chain alkyl group or a branched-chain alkyl group. For example, the alkyl group may be an ethyl group or a tert-butyl group. In the cation represented by General Formula (G0), it is preferable that R⁵ do not have an oxygen-oxygen bond (peroxide). An oxygen-oxygen single bond extremely easily breaks and is reactive; thus, the cation having the bond might be explosive. Therefore, an ionic liquid containing a cation having an oxygen-oxygen bond is not suitable for a power storage device.

A compound used for the power storage device of one embodiment of the present invention includes an anion and a cation represented by General Formula (G1).

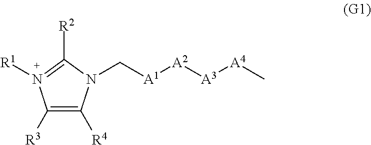

In General Formula (G1), R¹ represents an alkyl group having 1 to 4 carbon atoms, R² to R⁴ each independently represent a hydrogen atom or an alkyl group having 1 to 4 carbon atoms, A¹ to A⁴ each independently represent a methylene group or an oxygen atom, and at least one of A¹ to A⁴ represents an oxygen atom.

When a substituent (including substituents represented by A¹ to A⁴ in General Formula (G1)) is bonded to nitrogen of the imidazolium cation, the cation in the ionic liquid has sterically bulky structure and thus side reactions (e.g., cation insertion into graphite and decomposition of the nonaqueous solvent during charging, and gas generation associated with the insertion and the decomposition) in a battery can be suppressed. However, the viscosity of the ionic liquid is likely to be enhanced as the number of carbon atoms in A¹ to A⁴ is larger; therefore, it is preferable to determine the number of carbon atoms in the straight chain in accordance with desirable charge and discharge efficiency and desirable viscosity. It is preferable that the substituent represented by A¹ to A⁴ do not have an oxygen-oxygen bond (peroxide). An oxygen-oxygen single bond extremely easily breaks and is reactive; thus, the cation having the bond might be explosive. Therefore, an ionic liquid containing a cation having an oxygen-oxygen bond is not suitable for a power storage device.

An anion contained in the ionic liquid is a monovalent anion which forms the ionic liquid with the imidazolium cation. As the anion, the one described above can be used.

The anion in the ionic liquid is preferably a bis(fluorosulfonyl)amide anion that is a monovalent amide anion. An ionic liquid including a bis(fluorosulfonyl)amide anion and a cation can have high conductivity and relative low viscosity, and a storage device including the ionic liquid and using graphite for a negative electrode can be charged and discharged.

A compound used for the power storage device of one embodiment of the present invention includes an anion and a cation represented by General Formula (G2).

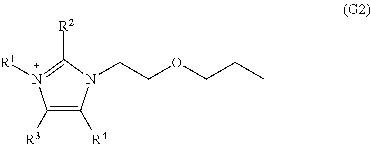

In General Formula (G2), R¹ represents an alkyl group having 1 to 4 carbon atoms, R² to R⁴ each independently represent a hydrogen atom or an alkyl group having 1 to 4 carbon atoms.

An anion contained in the ionic liquid is a monovalent anion which forms the ionic liquid with the imidazolium cation. As the anion, the one described above can be used.

The anion in the ionic liquid is preferably a bis(fluorosulfonyl)amide anion that is a monovalent amide anion.

A compound used for the power storage device of one embodiment of the present invention includes an anion and a cation represented by General Formula (G3).

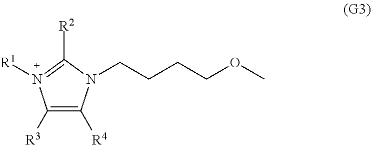

In General Formula (G3), R¹ represents an alkyl group having 1 to 4 carbon atoms, R² to R⁴ each independently represent a hydrogen atom or an alkyl group having 1 to 4 carbon atoms.

An anion contained in the ionic liquid is a monovalent anion which forms the ionic liquid with the imidazolium cation. As the anion, the one described above can be used.

The anion in the ionic liquid is preferably a bis(fluorosulfonyl)amide anion that is a monovalent amide anion.

Specific examples of the cation represented by General Formula (G0) include Structural Formulae (101) to (143), Structural Formulae (201) to (227), Structural Formulae (301) to (304), Structural Formulae (401) to (427), Structural Formulae (501) to (504), Structural Formulae (601) to (604), Structural Formulae (701) to (704), Structural Formulae (801) to (804), and Structural Formulae (901) to (913).

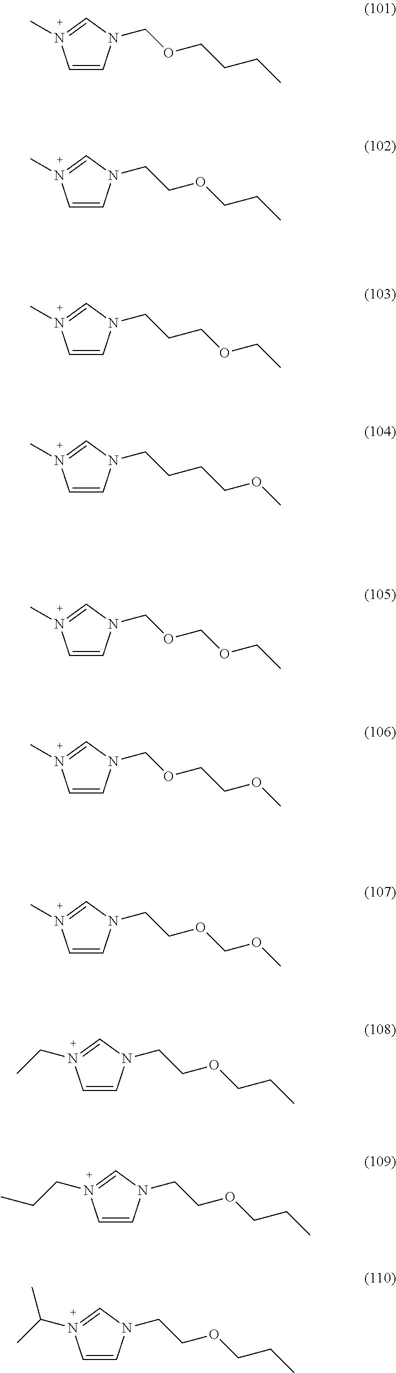

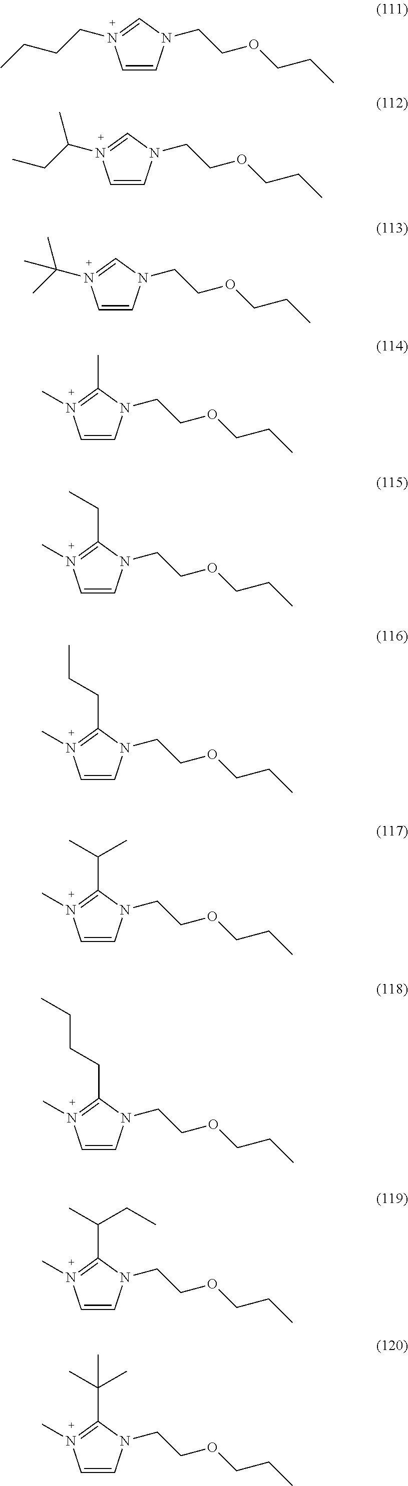

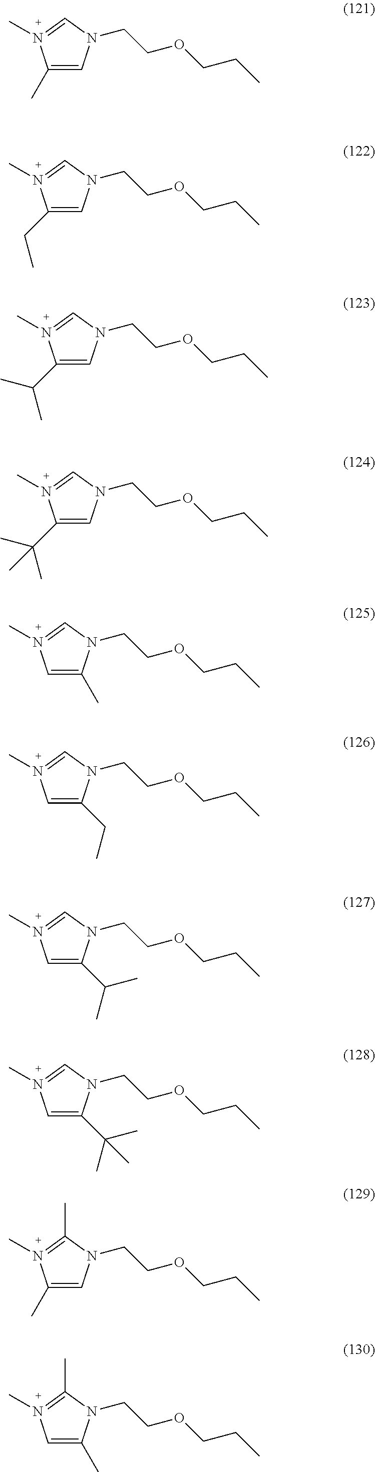

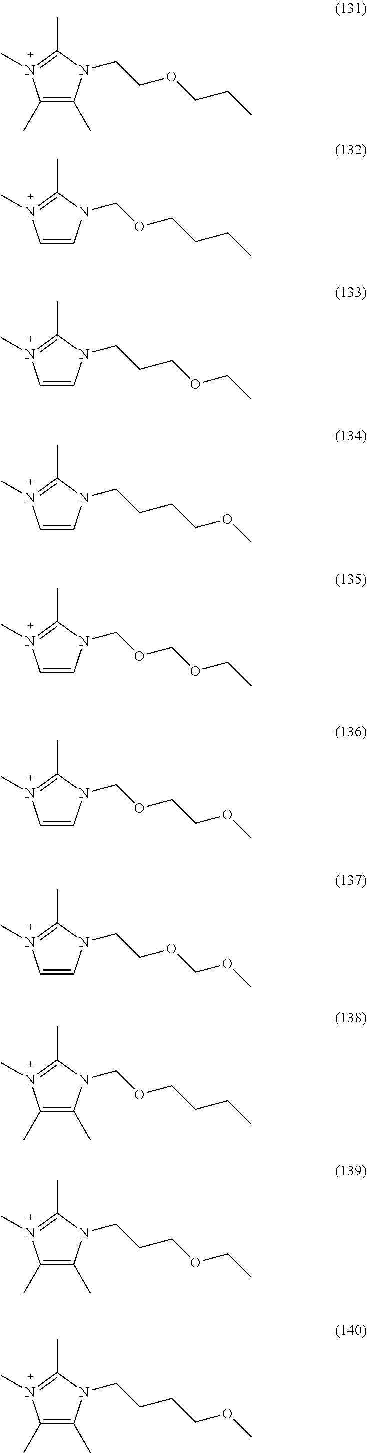

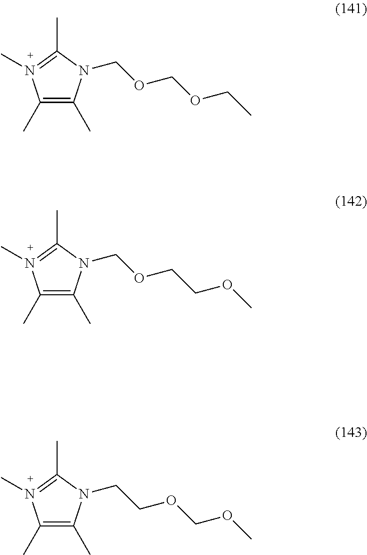

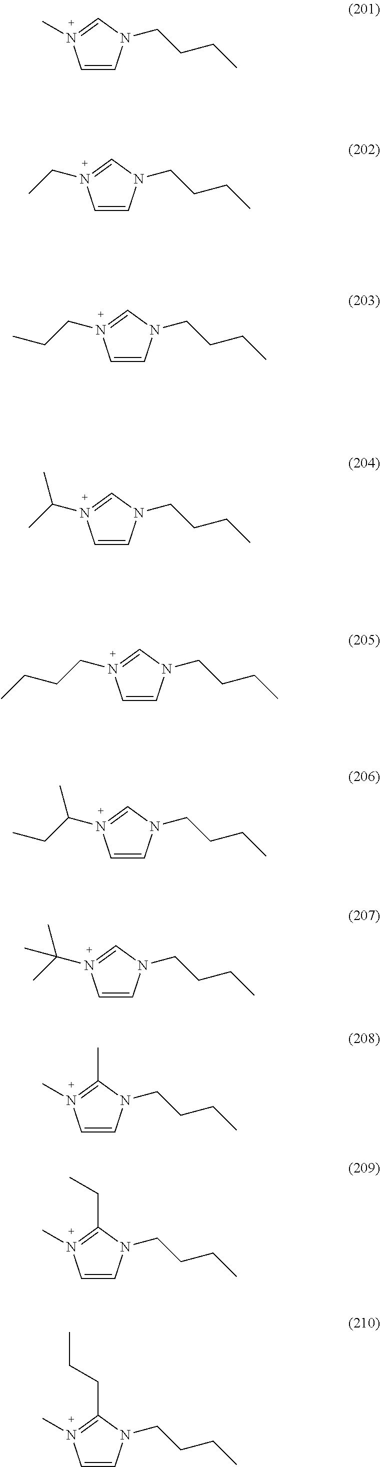

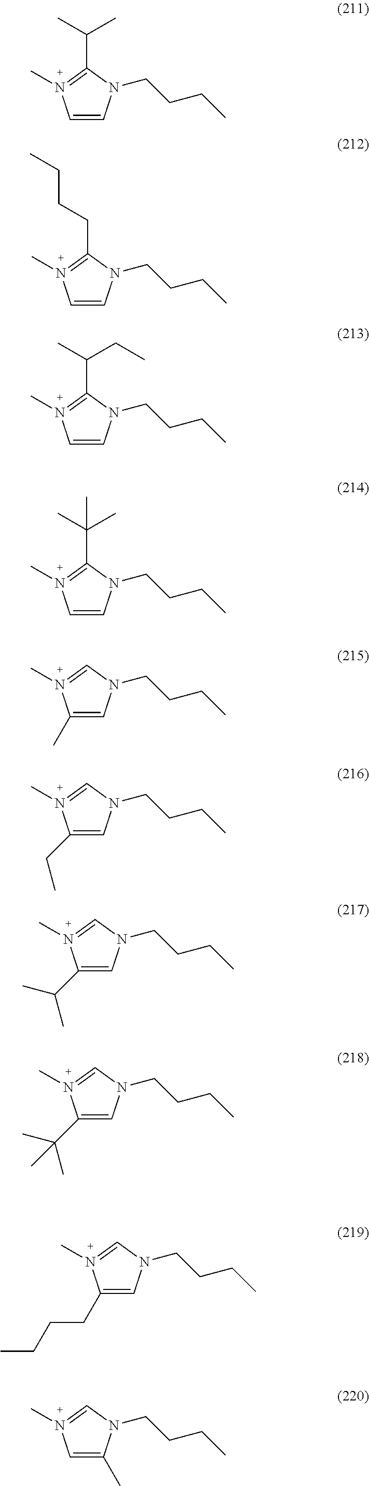

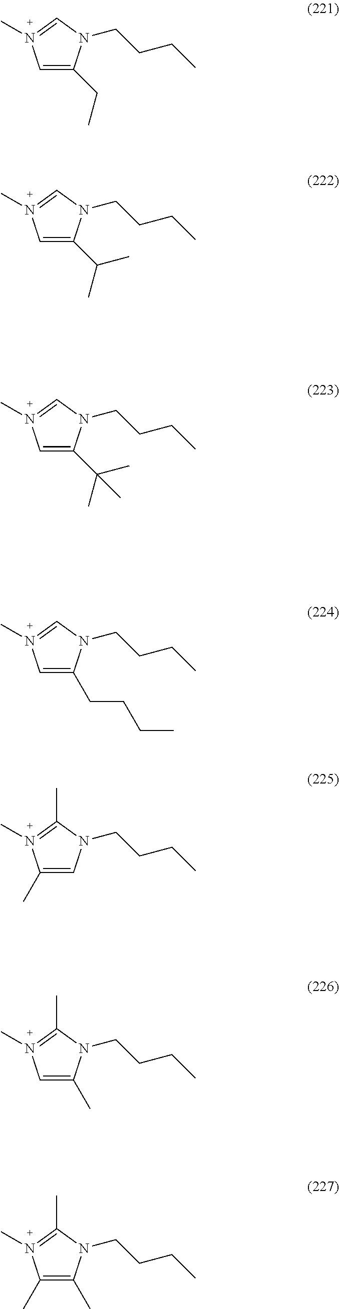

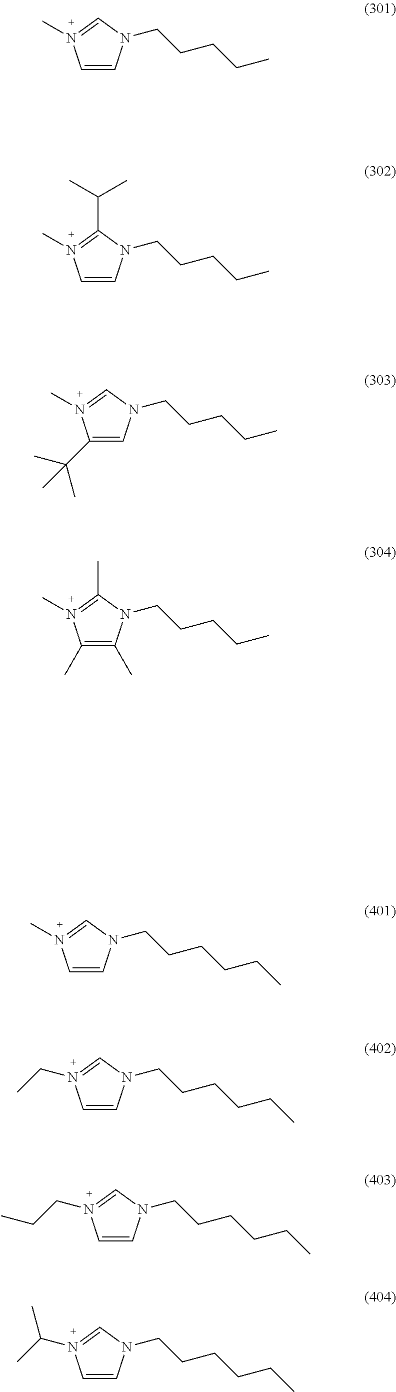

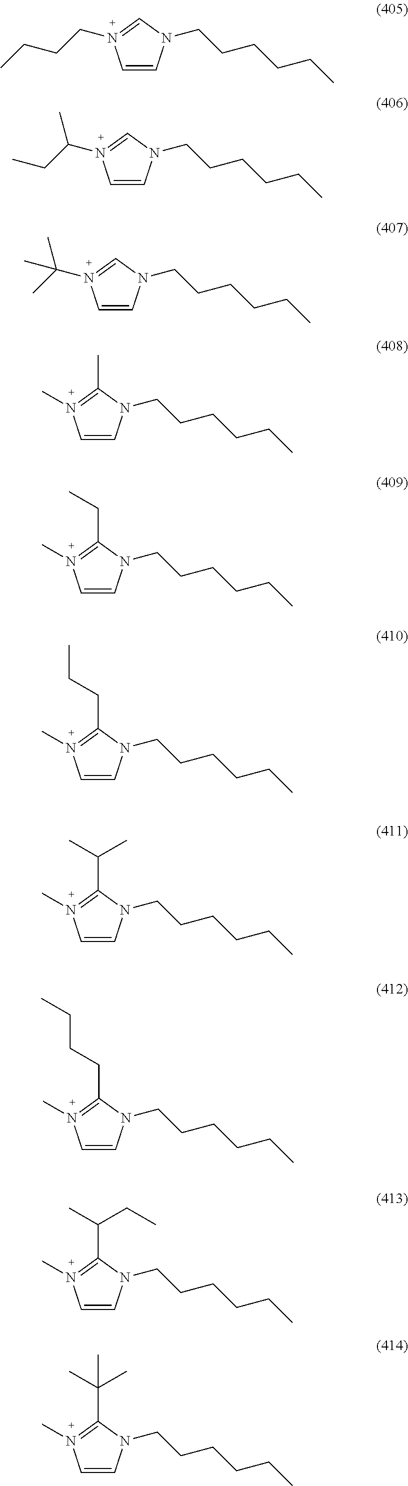

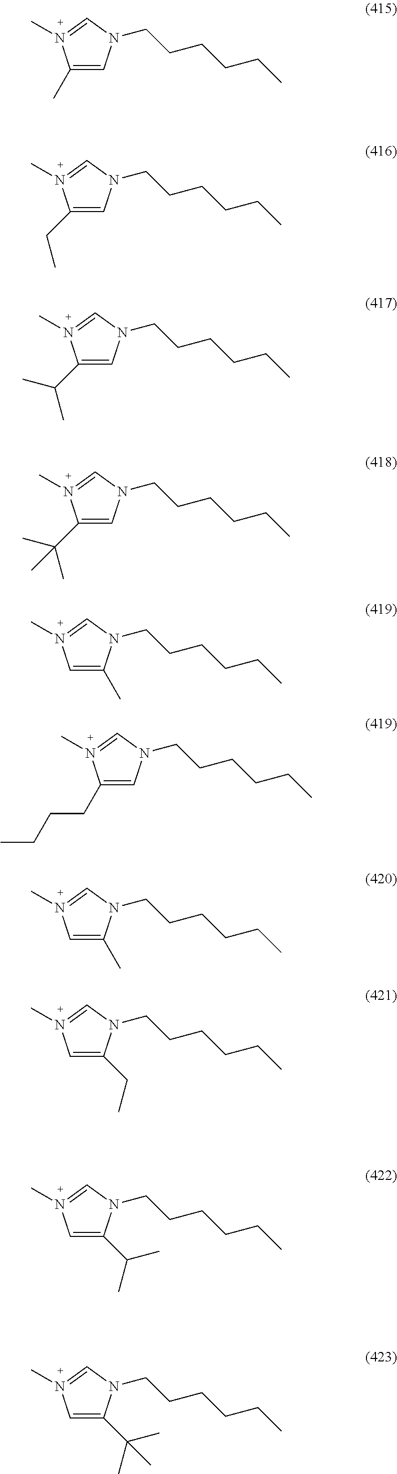

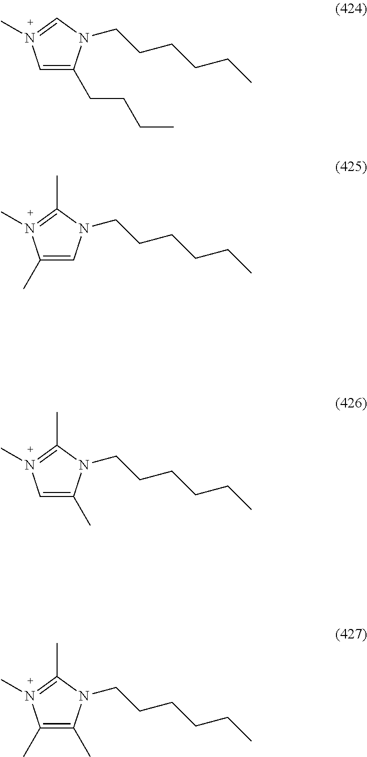

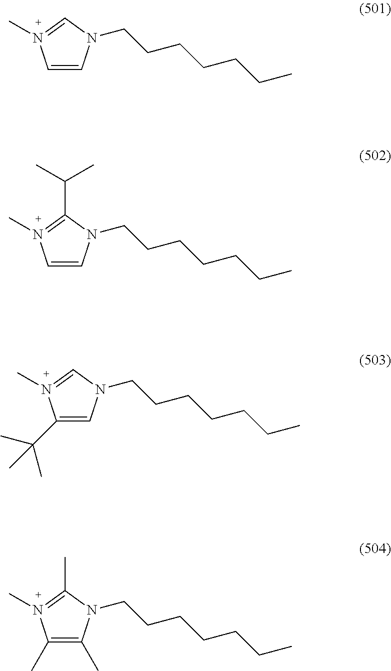

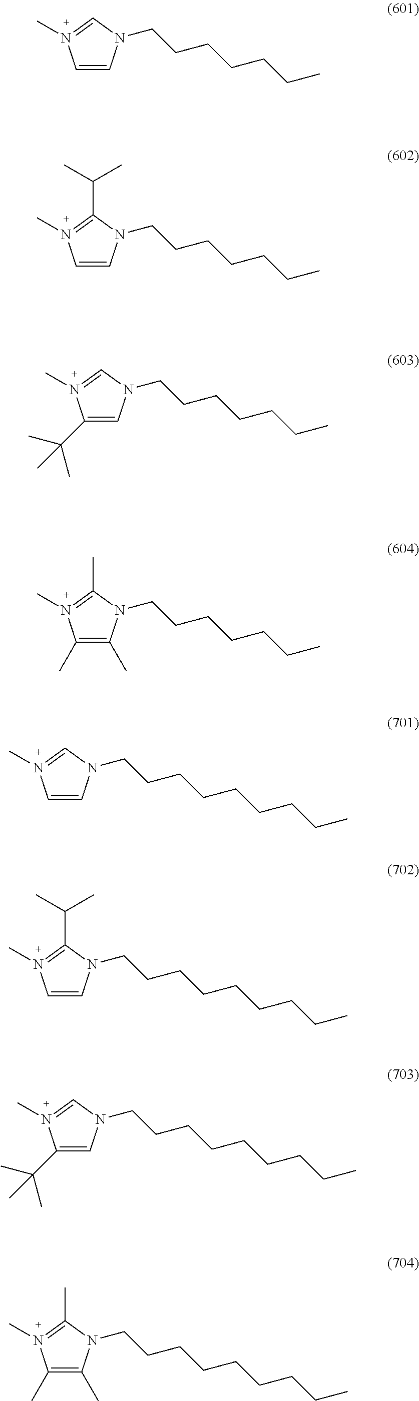

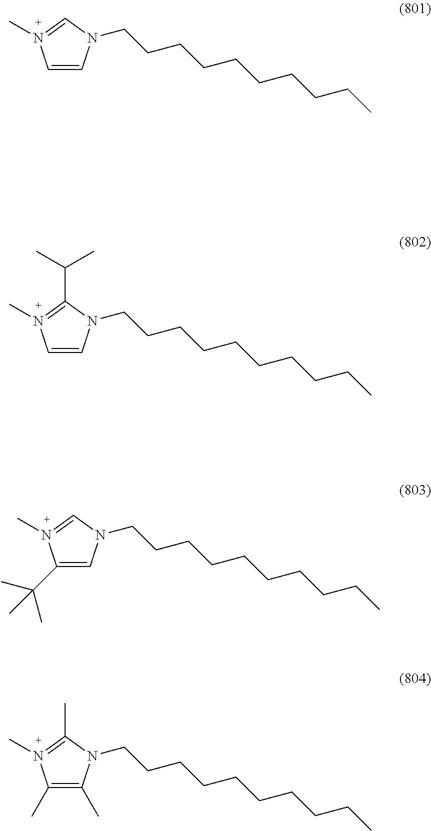

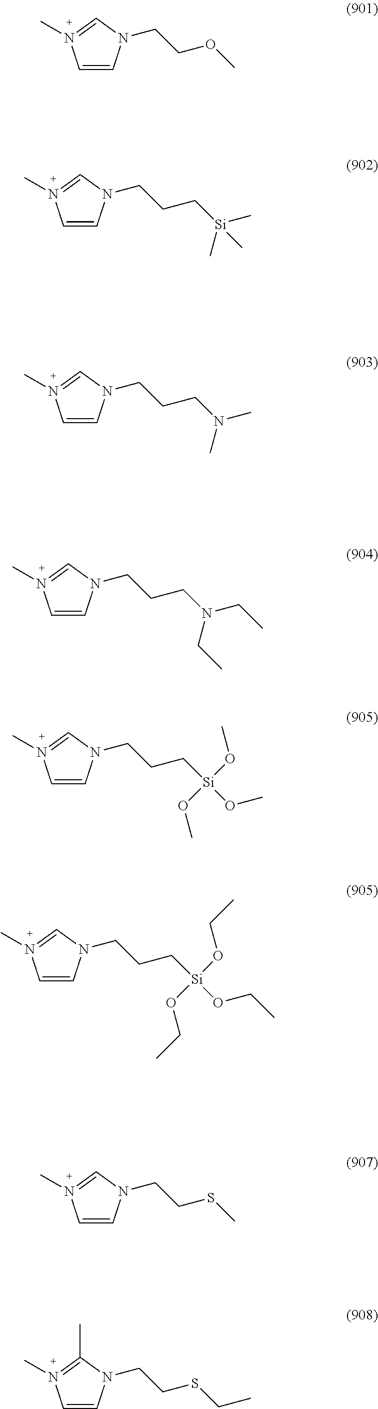

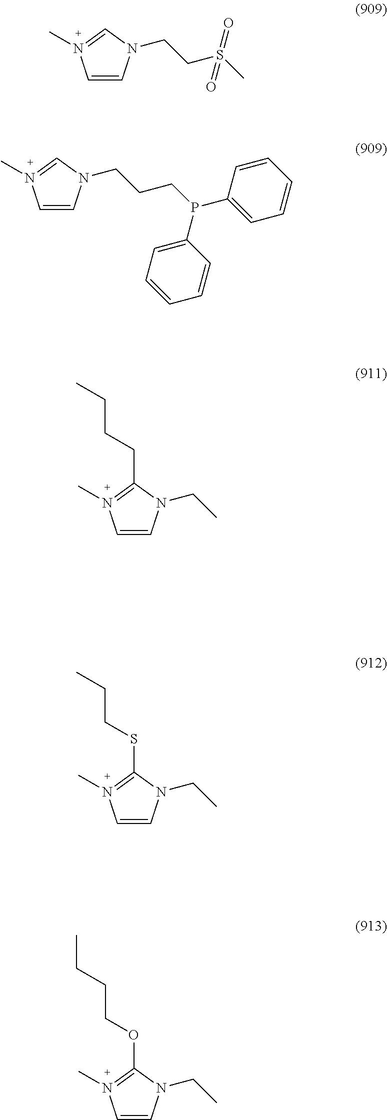

In the power storage device of one embodiment of the present invention, the ionic liquid may include any of stereoisomers represented by Structural Formulae (101) to (143), Structural Formulae (201) to (227), Structural Formulae (301) to (304), Structural Formulae (401) to (427), Structural Formulae (501) to (504), Structural Formulae (601) to (604), Structural Formulae (701) to (704), Structural Formulae (801) to (804), and Structural Formulae (901) to (913). Isomers are different compounds with the same molecular formula. Stereoisomers are isomers in which the geometrical positioning of atoms in space differs but the bond structure is the same. Thus, in this specification and the like, the term “stereoisomers” include enantiomers, geometric (cis-trans) isomers, and diastereomers which include two or more chiral centers and are not enantiomers.

Structural Formulae shown above are conjugated cyclic compounds. A conjugated system is a system of connected p-orbitals with delocalized electrons in compounds (electrons spread across the conjugated system) with alternating single and multiple bonds, which increases the stability. For example, the following two structural formulae are the same compounds with different positions of delocalized electrons.

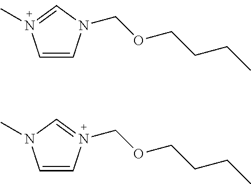

Furthermore, a plurality of ionic liquids may be used in the nonaqueous solvent included in the power storage device of one embodiment of the present invention, for example. As the plurality of ionic liquids, the ionic liquid represented by General Formula (G0) and the ionic liquid represented by General Formula (G0) may be used, for example. A nonaqueous solvent including a plurality of ionic liquids has a lower freezing point than a nonaqueous solvent including an ionic liquid in some cases. Thus, the use of a nonaqueous solvent including a plurality of ionic liquids enables a power storage device to be operated in a low-temperature environment in some cases. In that case, a power storage device that can be operated in a wide temperature range can be fabricated.

Furthermore, the reduction potential of the ionic liquid included in the nonaqueous solvent included in the power storage device of one embodiment of the present invention is preferably lower than the oxidation-reduction potential of lithium (Li/Li⁺), which is a typical low potential negative electrode material.

In the case where at least one of R¹ to R⁴ in the cation represented by any one of General Formulae (G0) to (G3) is an alkyl group having 1 to 4 carbon atoms, the number of carbon atoms is preferably small. An alkyl group having a small number of carbon atoms allows an ionic liquid to have low viscosity, resulting in a reduction in the viscosity of the nonaqueous solvent included in the power storage device of one embodiment of the present invention.

The oxidation potential of the ionic liquid changes depending on anionic species. Thus, in order to obtain an ionic liquid with high oxidation potential, the anion in the ionic liquid included in the nonaqueous solvent of the power storage device of one embodiment of the present invention is preferably a monovalent anion selected from (CₙF₂ₙ₊₁SO₂)₂N⁻ (n=0 to 3), CF₂(CF₂SO₂)₂N⁻, and (CₘF₂ₘ₊₁SO₃)⁻ (m=0 to 4). Note that the high oxidation potential means an improvement in oxidation resistance (also referred to as stability against oxidation). The oxidation resistance is improved by the interaction between a cation having a substituent and the anion described above.

Thus, by using the ionic liquid having improved oxidation resistance and causing less side reactions for the nonaqueous solvent of the power storage device of one embodiment of the present invention, decomposition of the nonaqueous solvent (specifically, a nonaqueous electrolyte including the nonaqueous solvent) due to charge and discharge can be suppressed. Furthermore, by decreasing the viscosity of the nonaqueous solvent of the power storage device of one embodiment of the present invention (specifically, the nonaqueous electrolyte including the nonaqueous solvent), the ion conductivity of the nonaqueous solvent can be improved. Thus, the use of the nonaqueous solvent of the power storage device of one embodiment of the present invention enables a power storage device with favorable charging and discharging rate characteristics to be manufactured.

An alkali metal salt that can be used in a nonaqueous electrolyte of the power storage device of one embodiment of the present invention is, for example, an alkali metal salt that contains alkali metal ions or alkaline-earth metal ions. Examples of the alkali metal ion include a lithium ion, a sodium ion, and a potassium ion. Examples of the alkaline earth metal ion include a calcium ion, a strontium ion, and a barium ion. Note that in this embodiment, a lithium salt including lithium ions is used as the salt. Examples of the lithium salt include lithium chloride (LiCl), lithium fluoride (LiF), lithium perchlorate (LiClO₄), lithium tetrafluoroborate (LiBF₄), LiAsF₆, LiPF₆, Li(CF₃SO₃), Li(FSO₂)₂N (what is called LiFSA), and Li(CF₃SO₂)₂N (what is called LiTFSA).

The nonaqueous solvent in which the cation in the ionic liquid becomes sterically bulky and thus side reactions are suppressed can have high conductivity and non-flammability.

Consequently, a nonaqueous electrolyte using the nonaqueous solvent and a power storage device using the nonaqueous electrolyte each have a high degree of safety and high performance.

Here, a method of synthesizing the ionic liquid described in this embodiment and represented by General Formula (G0) is described as an example.

### <Example of Method of Synthesizing Ionic Liquid Represented by General Formula (G0)>

A variety of reactions can be applied to the method of synthesizing the ionic liquid described in this embodiment. For example, the ionic liquid represented by the General Formula (G0) can be synthesized by a synthesis method described below. Here, an example is described referring to synthesis schemes. Note that the method of synthesizing the ionic liquid described in this embodiment is not limited to the following synthesis method.

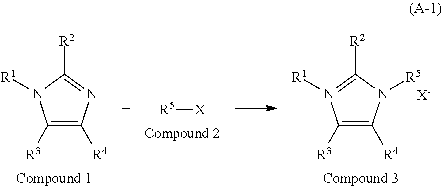

As shown in Scheme (A-1), an imidazole derivative (Compound 1) and a halide (Compound 2) are coupled to give an imidazolium salt (Compound 3). In Scheme (A-1), R¹ represents an alkyl group having 1 to 4 carbon atoms; R² to R⁴ each independently represent a hydrogen atom or an alkyl group having 1 to 4 carbon atoms; R⁵ represents a straight chain formed of four or more atoms and includes one or more of C, O, Si, N, S, and P; and X represents halogen.

Synthesis under Scheme (A-1) can be carried out with or without a solvent. Examples of the solvent that can be used in Scheme (A-1) include, but not limited to, alcohols such as ethanol and methanol, nitriles such as acetonitrile, ethers such as diethyl ether, tetrahydrofuran, and 1,4-dioxane.

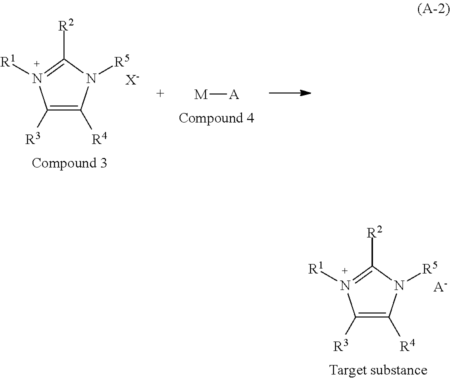

As shown in Scheme (A-2), ion exchange between the imidazolium salt (Compound 3) and a desired metallic salt (Compound 4) containing A is performed to give the target substance. In Scheme (A-2), R¹ represents an alkyl group having 1 to 4 carbon atoms; R² to R⁴ each independently represent a hydrogen atom or an alkyl group having 1 to 4 carbon atoms; R⁵ represents a straight chain formed of four or more atoms and includes one or more of C, O, Si, N, S, and P; and X represents halogen.

In Scheme (A-2), A is any one of a monovalent amide anion, a monovalent methide anion, a fluorosulfonate anion (SO₃F⁻), a perfluoroalkylsulfonate anion, a tetrafluoroborate anion (BF₄⁻), a perfluoroalkylborate anion, a hexafluorophosphate anion (PF₆⁻), and a perfluoroalkylphosphate anion. Note that the solvent that can be used is not limited thereto.

In Scheme (A-2), M represents an alkali metal or the like. Examples of the alkali metal are, but not limited to, potassium, sodium, and lithium.

Synthesis under Scheme (A-2) can be carried out with or without a solvent. Examples of the solvent that can be used in Scheme (A-2) include, but not limited to, water, alcohols such as ethanol and methanol, nitriles such as acetonitrile, ethers such as diethyl ether, tetrahydrofuran, and 1,4-dioxane.

Next, a method of synthesizing the ionic liquid described in this embodiment and represented by General Formula (G1) is described as an example.

### <Example of Method of Synthesizing Ionic Liquid Represented by General Formula (G1)>

A variety of reactions can be applied to the method of synthesizing the ionic liquid described in this embodiment. For example, the ionic liquid represented by the General Formula (G1) can be synthesized by a synthesis method described below. Here, an example is described referring to synthesis schemes. Note that the method of synthesizing the ionic liquid described in this embodiment is not limited to the following synthesis method.

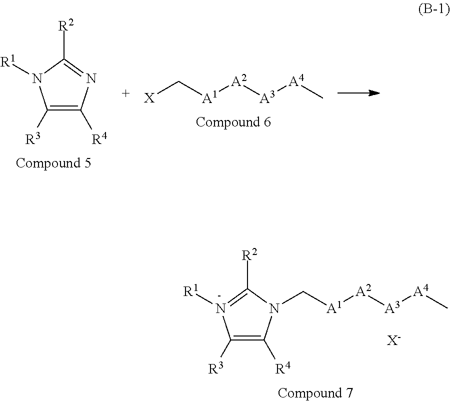

As shown in Scheme (B-1), an imidazole derivative (Compound 5) and a halide of alkoxyalkyl (Compound 6) are coupled to give an imidazolium salt (Compound 7). In Scheme (B-1), A¹ to A⁴ each independently represent a methylene group or an oxygen atom, and at least one of A¹ to A⁴ represents an oxygen atom; R¹ represents an alkyl group having 1 to 4 carbon atoms; R² to R⁴ each independently represent a hydrogen atom or an alkyl group having 1 to 4 carbon atoms; and X represents halogen.

Synthesis under Scheme (B-1) can be carried out with or without a solvent. Examples of the solvent that can be used in Scheme (B-1) include, but not limited to, alcohols such as ethanol and methanol, nitriles such as acetonitrile, ethers such as diethyl ether, tetrahydrofuran, and 1,4-dioxane.

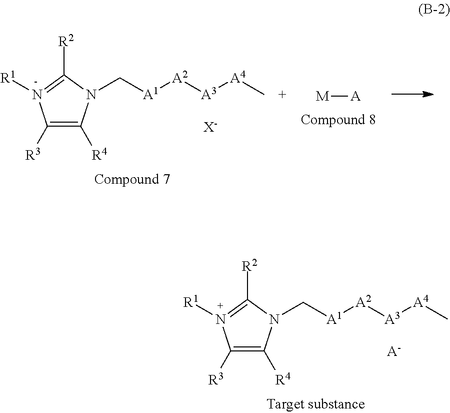

As shown in Scheme (B-2), ion exchange between the imidazolium salt (Compound 7) and a desired metallic salt (Compound 8) containing A is performed to give the target substance. In Scheme (B-2), A¹ to A⁴ each independently represent a methylene group or an oxygen atom, and at least one of A¹ to A⁴ represents an oxygen atom; R¹ represents an alkyl group having 1 to 4 carbon atoms; R² to R⁴ each independently represent a hydrogen atom or an alkyl group having 1 to 4 carbon atoms; and X represents halogen.

In Scheme (B-2), A is any one of a monovalent amide anion, a monovalent methide anion, a fluorosulfonate anion (SO₃F⁻), a perfluoroalkylsulfonate anion, a tetrafluoroborate anion (BF₄⁻), a perfluoroalkylborate anion, a hexafluorophosphate anion (PF₆⁻), and a perfluoroalkylphosphate anion. Note that the solvent that can be used is not limited thereto.

In Scheme (B-2), M represents an alkali metal or the like. Examples of the alkali metal are, but not limited to, potassium, sodium, and lithium.

Synthesis under Scheme (B-2) can be carried out with or without a solvent.

Examples of the solvent that can be used in Scheme (B-2) include, but not limited to, water, alcohols such as ethanol and methanol, nitriles such as acetonitrile, ethers such as diethyl ether, tetrahydrofuran, and 1,4-dioxane.

In the above-mentioned manner, the nonaqueous solvent for the power storage device of one embodiment of the present invention can be formed. The nonaqueous solvent of one embodiment of the present invention can have flame retardance. Furthermore, the nonaqueous solvent of one embodiment of the present invention can have high ionic conductivity. Therefore, a power storage device using the nonaqueous solvent of one embodiment of the present invention can have a high degree of safety and favorable charge and discharge rate characteristics.

This embodiment can be implemented in appropriate combination with any of the structures described in the other embodiments.

### Embodiment 2

### Coin-Type Storage Battery

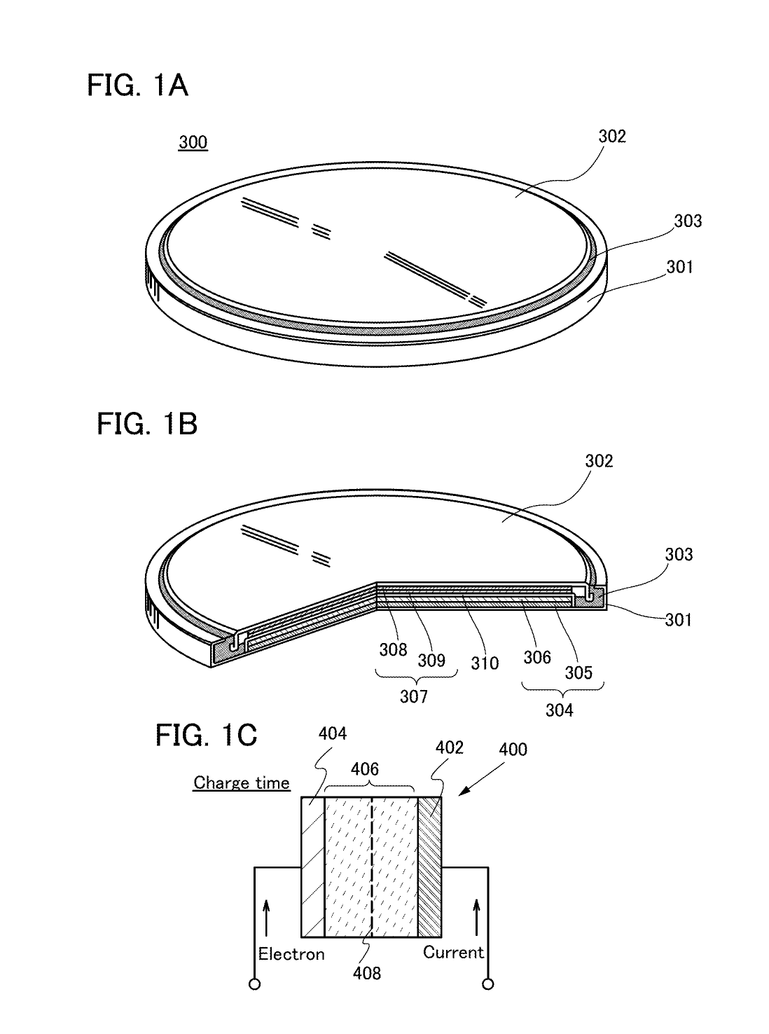

FIG. 1A is an external view of a coin-type (single-layer flat type) storage battery, and FIG. 1B is a cross-sectional view thereof.

In a coin-type storage battery 300, a positive electrode can 301 doubling as a positive electrode terminal and a negative electrode can 302 doubling as a negative electrode terminal are insulated from each other and sealed by a gasket 303 made of polypropylene or the like. A positive electrode 304 includes a positive electrode current collector 305 and a positive electrode active material layer 306 provided in contact with the positive electrode current collector 305. The positive electrode active material layer 306 may further include a binder for increasing adhesion of positive electrode active materials, a conductive additive for increasing the conductivity of the positive electrode active material layer, and the like in addition to the active materials. As the conductive additive, a material that has a large specific surface area is preferably used; for example, acetylene black (AB) can be used. Alternatively, a carbon material such as a carbon nanotube, graphene, or fullerene can be used. Graphene is flaky and has an excellent electric characteristic of high conductivity and excellent physical properties of high flexibility and high mechanical strength. Thus, the use of graphene as the conductive additive can increase contact points and the contact area of active materials. Note that graphene in this specification includes single-layer graphene and multilayer graphene including two or more and a hundred or less layers. Single-layer graphene refers to a one-atom-thick sheet of carbon molecules having π bonds.

A negative electrode 307 includes a negative electrode current collector 308 and a negative electrode active material layer 309 provided in contact with the negative electrode current collector 308. The negative electrode active material layer 309 may further include a binder for increasing adhesion of negative electrode active materials, a conductive additive for increasing the conductivity of the negative electrode active material layer, and the like. A separator 310 and an electrolyte (not illustrated) are provided between the positive electrode active material layer 306 and the negative electrode active material layer 309.

Gallium is used as a negative electrode active material in the negative electrode active material layer 309. For example, copper is used as the negative electrode current collector 308, and copper and gallium are alloyed. The adhesion between the current collector and the active material (gallium) is improved by the alloying, and thus deterioration due to expansion and contraction or deterioration of a secondary battery due to deformation (e.g., bending) can be prevented.

The current collectors 305 and 308 can each be formed with a highly conductive material which is not alloyed with a carrier ion of lithium among other elements, such as a metal typified by stainless steel, gold, platinum, zinc, iron, nickel, copper, aluminum, titanium, and tantalum or an alloy thereof. Alternatively, an aluminum alloy to which an element which improves heat resistance, such as silicon, titanium, neodymium, scandium, and molybdenum, is added can be used. Still alternatively, a metal element which forms silicide by reacting with silicon can be used. Examples of the metal element which forms silicide by reacting with silicon include zirconium, titanium, hafnium, vanadium, niobium, tantalum, chromium, molybdenum, tungsten, cobalt, nickel, and the like. The current collectors can each have a foil-like shape, a plate-like shape (sheet-like shape), a net-like shape, a cylindrical shape, a coil shape, a punching-metal shape, an expanded-metal shape, or the like as appropriate. The current collectors each preferably have a thickness of 10 μm to 30 μm inclusive.

An oxide or composite oxide having an olivine crystal structure, a layered rock-salt crystal structure, or a spinel crystal structure or the like can be used for a positive electrode active material of the positive electrode active material layer 306. For the positive electrode active material, compounds such as LiFeO₂, LiCoO₂, LiNiO₂, LiMn₂O₄, V₂O₅, Cr₂O₅, and MnO₂ can be used.

Alternatively, a complex material (LiMPO₄ (general formula) (M is one or more of Fe(II), Mn(II), Co(II), and Ni(II))) can be used. Typical examples of LiMPO₄ (general formula) which can be used as a material are lithium compounds such LiFePO₄, LiNiPO₄, LiCoPO₄, LiMnPO₄, LiFeₐNi_{b}PO₄, LiFeₐCo_{b}PO₄, LiFeₐMn_{b}PO₄, LiNiₐCo_{b}PO₄, LiNiₐMn_{b}PO₄ (a+b≦1, 0<a<1, and 0<b<1), LiFe_{c}Ni_{d}CoₑPO₄, LiFe_{c}Ni_{d}MnₑPO₄, LiNi_{c}Co_{d}MnₑPO₄ (c+d+e≦1, 0<c<1, 0<d<1, and 0<e<1), and LiFe_{f}Ni_{g}CoₕMnᵢPO₄ (f+g+h+i≦1, 0<f<1, 0<g<1, 0<h<1, and 0<i<1).

Alternatively, a complex material such as Li₍₂₋ⱼ₎MSiO₄ (general formula) (M is one or more of Fe(II), Mn(II), Co(II), and Ni(II); 0≦j≦2) can be used. Typical examples of the general formula Li₍₂₋ⱼ₎MSiO₄ which can be used as a material are lithium compounds such as Li₂₋ⱼ₎FeSiO₄, Li₍₂₋ⱼ₎NiSiO₄, Li₍₂₋ⱼ₎CoSiO₄, Li₍₂₋ⱼ₎MnSiO₄, Li₍₂₋ⱼ₎FeₖNiₗSiO₄, Li₍₂₋ⱼ₎FeₖCOₗSiO₄, Li₍₂₋ⱼ₎FeₖMnₗSiO₄, Li₍₂₋ⱼ₎NiₖCoₗSiO₄, Li₍₂₋ⱼ₎NiₖMnₗSiO₄ (k+l≦1, 0<k<1, and 0<l<1), Li₍₂₋ⱼ₎FeₘNiₙCo_{q}SiO₄, Li₍₂₋ⱼ₎FeₘNiₙMn_{q}SiO₄, Li₍₂₋ⱼ₎NiₘCoₙMn_{q}SiO₄ (m+n+q≦1, 0<m<1, 0<n<1, and 0<q<1), and Li₍₂₋ⱼ₎FeᵣNiₛCoₜMnᵤSiO₄ (r+s+t+u≦1, 0<r<1, 0<s<1, 0<t<1, and 0<u<1).

Alternatively, a nasicon compound expressed by AₓM₂(XO₄)₃ (general formula) (A=Li, Na, or Mg, M=Fe, Mn, Ti, V, Nb, or Al, X═S, P, Mo, W, As, or Si) can be used as the positive electrode active material. Examples of the nasicon compound are Fe₂(MnO₄)₃, Fe₂(SO₄)₃, and Li₃Fe₂(PO₄)₃. Still alternatively, a compound expressed by the general formula Li₂MPO₄F, Li₂MP₂O₇, or Li₅MO₄ (M=Fe or Mn), a perovskite fluoride such as NaFeF₃ or FeF₃, a metal chalcogenide (a sulfide, a selenide, or a telluride) such as TiS₂ or MoS₂, an oxide with an inverse spinel crystal structure such as LiMVO₄, a vanadium oxide (e.g., V₂O₅, V₆O₁₃, or LiV₃O₈), a manganese oxide, an organic sulfur compound, or the like can be used as the positive electrode active material.

In the case where carrier ions are alkali metal ions other than lithium ions or alkaline-earth metal ions, the following may be used as the positive electrode active material: an alkali metal (e.g., sodium or potassium), or an alkaline-earth metal (e.g., calcium, strontium, or barium, beryllium, or magnesium).

As the separator 310, an insulator such as cellulose (paper), polyethylene with pores, and polypropylene with pores can be used.

As an electrolyte of an electrolyte solution, a material which contains carrier ions is used. Typical examples of the electrolyte are lithium salts such as LiPF₆, LiClO₄, LiAsF₆, LiBF₄, LiCF₃SO₃, Li(CF₃SO₂)₂N, and Li(C₂F₅SO₂)₂N. One of these electrolytes may be used alone or two or more of them may be used in an appropriate combination and in an appropriate ratio.

Note that when carrier ions are alkali metal ions other than lithium ions, alkaline-earth metal ions, beryllium ions, or magnesium ions, instead of lithium in the above lithium salts, an alkali metal (e.g., sodium and potassium), an alkaline-earth metal (e.g., calcium, strontium, barium, beryllium, and magnesium) may be used for the electrolyte.

As a solvent of the electrolytic solution, a material in which carrier ions can transfer is used. As the solvent of the electrolytic solution, an aprotic organic solvent is preferably used. Typical examples of aprotic organic solvents include ethylene carbonate (EC), propylene carbonate, dimethyl carbonate, diethyl carbonate (DEC), γ-butyrolactone, acetonitrile, dimethoxyethane, tetrahydrofuran, and the like, and one or more of these materials can be used. When a gelled high-molecular material is used as the solvent of the electrolytic solution, safety against liquid leakage is improved. Furthermore, the storage battery can be thinner and more lightweight. Typical examples of gelled high-molecular materials include a silicone gel, an acrylic gel, an acrylonitrile gel, polyethylene oxide, polypropylene oxide, a fluorine-based polymer, and the like. Alternatively, the use of one or more of ionic liquids (room temperature molten salts) which have features of non-flammability and non-volatility as a solvent of the electrolytic solution can prevent the storage battery from exploding or catching fire even when the storage battery internally shorts out or the internal temperature increases owing to overcharging and others. An ionic liquid is a salt in the liquid state and has high ion mobility (conductivity). The ionic liquid includes a cation and an anion. As the ionic liquid, the ionic liquid described in Embodiment 1 can be used.

Instead of the electrolytic solution, a solid electrolyte including an inorganic material such as a sulfide-based inorganic material or an oxide-based inorganic material, or a solid electrolyte including a macromolecular material such as a polyethylene oxide (PEO)-based macromolecular material may alternatively be used. When the solid electrolyte is used, a separator and a spacer are not necessary. Furthermore, the battery can be entirely solidified; therefore, there is no possibility of liquid leakage and thus the safety of the battery is dramatically increased.

For the positive electrode can 301 and the negative electrode can 302, a metal having corrosion resistance to an electrolytic solution, such as aluminum, or titanium, an alloy of such metals, or an alloy of such a metal and another metal (stainless steel or the like) can be used. Alternatively, it is preferable to cover the positive electrode can 301 and the negative electrode can 302 with aluminum or the like in order to prevent corrosion due to the electrolytic solution. The positive electrode can 301 and the negative electrode can 302 are electrically connected to the positive electrode 304 and the negative electrode 307, respectively. When an exterior body containing a resin material is used instead of the positive electrode can 301 formed with metal or the negative electrode can 302 formed with metal, the coin-type storage battery 300 can have flexibility. Note that in the case where the exterior body containing a resin material is used, a conductive material is used for a portion connected to the outside.

The negative electrode 307, the positive electrode 304, and the separator 310 are immersed in the electrolytic solution. Then, as illustrated in FIG. 1B, the positive electrode 304, the separator 310, the negative electrode 307, and the negative electrode can 302 are stacked in this order with the positive electrode can 301 positioned at the bottom, and the positive electrode can 301 and the negative electrode can 302 are subjected to pressure bonding with the gasket 303 interposed therebetween. In such a manner, the coin-type storage battery 300 can be manufactured.

Here, a current flow in charging a battery will be described with reference to FIG. 1C. When a battery using lithium is regarded as a closed circuit, lithium ions transfer and a current flows in the same direction. Note that in the battery using lithium, an anode and a cathode change places in charge and discharge, and an oxidation reaction and a reduction reaction occur on the corresponding sides; hence, an electrode with a high redox potential is called a positive electrode and an electrode with a low redox potential is called a negative electrode. For this reason, in this specification, the positive electrode is referred to as a “positive electrode” and the negative electrode is referred to as a “negative electrode” in all the cases where charge is performed, discharge is performed, a reverse pulse current is supplied, and a charging current is supplied. The use of the terms “anode” and “cathode” related to an oxidation reaction and a reduction reaction might cause confusion because the anode and the cathode change places at the time of charging and discharging. Thus, the terms “anode” and “cathode” are not used in this specification. If the term “anode” or “cathode” is used, it should be mentioned that the anode or the cathode is which of the one at the time of charging or the one at the time of discharging and corresponds to which of a positive electrode or a negative electrode.

Two terminals in FIG. 1C are connected to a charger, and a storage battery 400 is charged. As the charge of the storage battery 400 proceeds, a potential difference between electrodes increases. The positive direction in FIG. 1C is the direction in which a current flows from one terminal outside the storage battery 400 to a positive electrode 402, flows from the positive electrode 402 to a negative electrode 404 in the storage battery 400, and flows from the negative electrode 404 to the other terminal outside the storage battery 400. In other words, a current flows in the direction of a flow of a charging current. Moreover, a separator 408 and an electrolyte 406 are provided between the positive electrode 402 and the negative electrode 404.

### [Cylindrical Storage Battery]

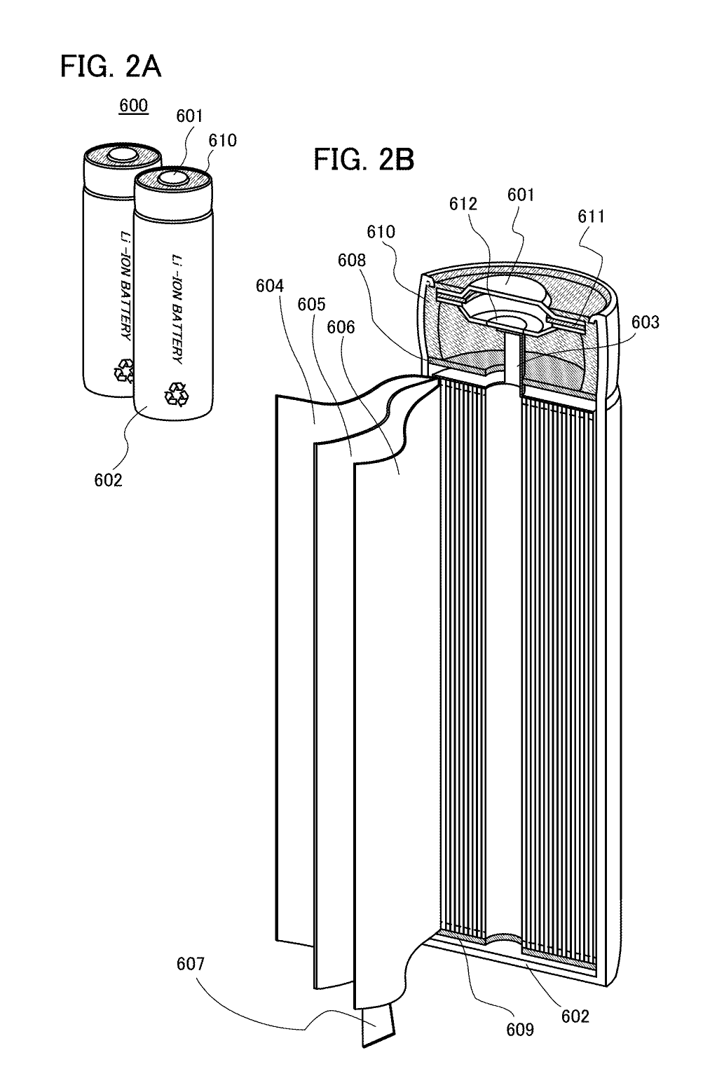

Next, an example of a cylindrical storage battery will be described with reference to FIGS. 2A and 2B. As illustrated in FIG. 2A, a cylindrical storage battery 600 includes a positive electrode cap (battery cap) 601 on the top surface and a battery can (outer can) 602 on the side surface and bottom surface. The positive electrode cap (battery cap) 601 and the battery can 602 are insulated from each other by a gasket (insulating gasket) 610.

FIG. 2B is a diagram schematically illustrating a cross section of the cylindrical storage battery. Inside the battery can 602 having a hollow cylindrical shape, a battery element in which a strip-like positive electrode 604 and a strip-like negative electrode 606 are wound with a stripe-like separator 605 interposed therebetween is provided. Although not illustrated, the battery element is wound around a center pin. One end of the battery can 602 is close and the other end thereof is open. For the battery can 602, a metal having corrosion resistance to an electrolytic solution, such as aluminum or titanium, an alloy of such a metal, or an alloy of such a metal and another metal (e.g., stainless steel) can be used. Alternatively, the battery can 602 is preferably covered with aluminum or the like in order to prevent corrosion caused by an electrolytic solution. Inside the battery can 602, the battery element in which the positive electrode, the negative electrode, and the separator are wound is provided between a pair of insulating plates 608 and 609 which face each other. Inside the battery can 602, the battery element in which the positive electrode, the negative electrode, and the separator are wound is interposed between a pair of insulating plates 608 and 609 which face each other. Furthermore, a nonaqueous electrolytic solution (not illustrated) is injected inside the battery can 602 provided with the battery element. As the nonaqueous electrolytic solution, a nonaqueous electrolytic solution which is similar to that of the above coin-type storage battery can be used. Note that when an exterior body including a resin material is used instead of the battery can 602 formed with metal, a flexible cylindrical storage battery can be manufactured. Note that in the case where an exterior body including a resin material is used, a conductive material is used for a portion connected to the outside.

Although the positive electrode 604 and the negative electrode 606 can be formed in a manner similar to that of the positive electrode and the negative electrode of the coin-type storage battery described above, the difference lies in that, since the positive electrode and the negative electrode of the cylindrical storage battery are wound, active materials are formed on both sides of the current collectors. A positive electrode terminal (positive electrode current collecting lead) 603 is connected to the positive electrode 604, and a negative electrode terminal (negative electrode current collecting lead) 607 is connected to the negative electrode 606. Both the positive electrode terminal 603 and the negative electrode terminal 607 can be formed with a metal material such as aluminum. The positive electrode terminal 603 and the negative electrode terminal 607 are resistance-welded to a safety valve mechanism 612 and the bottom of the battery can 602, respectively. The safety valve mechanism 612 is electrically connected to the positive electrode cap 601 through a positive temperature coefficient (PTC) element 611. The safety valve mechanism 612 cuts off electrical connection between the positive electrode cap 601 and the positive electrode 604 when the internal pressure of the battery exceeds a predetermined threshold value. The PTC element 611, which serves as a thermally sensitive resistor whose resistance increases as temperature rises, limits the amount of current by increasing the resistance, in order to prevent abnormal heat generation. Note that barium titanate (BaTiO₃)-based semiconductor ceramic can be used for the PTC element.

### [Thin Storage Battery]

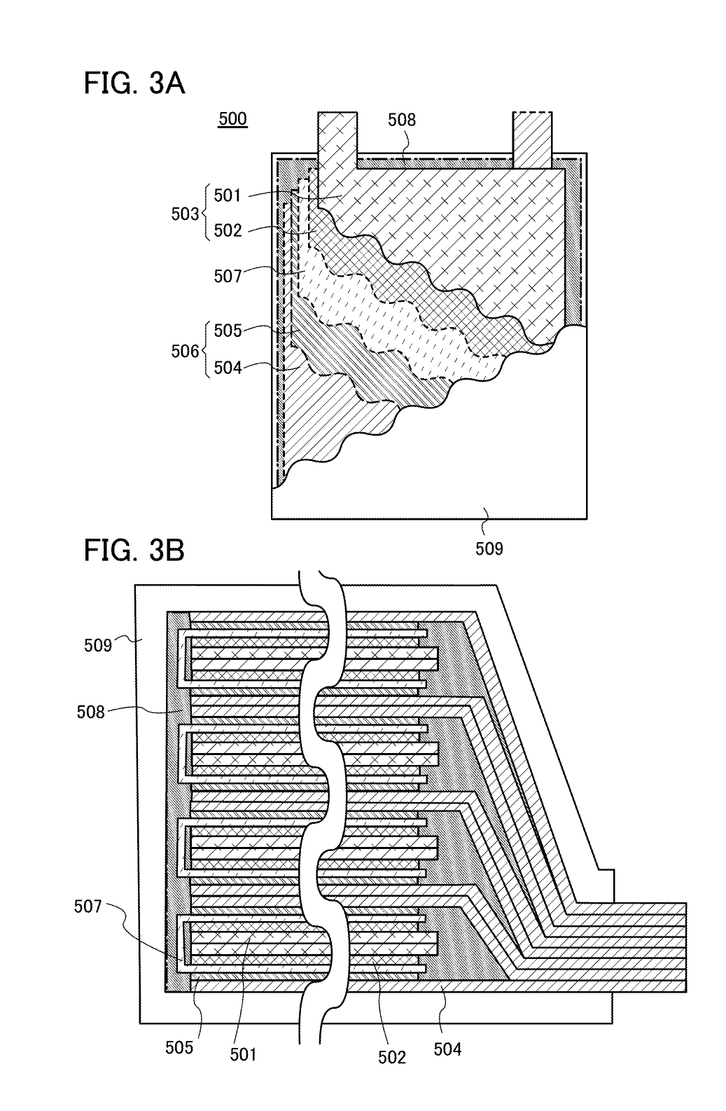

Next, an example of a thin storage battery will be described with reference to FIG. 3A. When a flexible thin storage battery is used in an electronic device at least part of which is flexible, the storage battery can be bent as the electronic device is bent.

A thin storage battery 500 illustrated in FIG. 3A includes a positive electrode 503 including a positive electrode current collector 501 and a positive electrode active material layer 502, a negative electrode 506 including a negative electrode current collector 504 and a negative electrode active material layer 505, a separator 507, an electrolytic solution 508, and an exterior body 509. The separator 507 is provided between the positive electrode 503 and the negative electrode 506 in the exterior body 509. The exterior body 509 is filled with the electrolytic solution 508.

In the thin storage battery 500 illustrated in FIG. 3A, the positive electrode current collector 501 and the negative electrode current collector 504 also serve as terminals for an electrical contact with an external portion. For this reason, each of the positive electrode current collector 501 and the negative electrode current collector 504 is arranged so that part of the positive electrode current collector 501 and part of the negative electrode current collector 504 are exposed to the outside the exterior body 509.

Alternatively, a lead electrode and the positive electrode current collector 501 or the negative electrode current collector 504 may be bonded to each other by ultrasonic welding, and instead of the positive electrode current collector 501 and the negative electrode current collector 504, part of the lead electrode may be exposed to the outside the exterior body 509.

As the exterior body 509 in the thin storage battery 500, for example, a film having a three-layer structure in which a highly flexible metal thin film of aluminum, stainless steel, copper, nickel, or the like is provided over a film formed of a material such as polyethylene, polypropylene, polycarbonate, ionomer, or polyamide, and an insulating synthetic resin film of a polyamide-based resin, a polyester-based resin, or the like is provided as the outer surface of the exterior body over the metal thin film can be used. For example, a stacked film including a resin film and a metal thin film may be used. The film including at least a resin film and a metal thin film is lightweight and has a high barrier property against moisture and a heat dissipation property; thus, the film is suitably used for a storage battery in a portable electronic device.

FIG. 3B illustrates an example of the cross-sectional structure of the thin storage battery 500. Although FIG. 3A illustrates an example of including two current collectors (i.e., a pair of current collectors) for simplicity, the actual battery includes three or more electrode layers.

The example in FIG. 3B includes 16 electrode layers. The thin storage battery 500 has flexibility even though including 16 electrode layers. In FIG. 3B, 8 negative electrode current collectors 504 and 8 positive electrode current collectors 501 are included. Note that FIG. 3B illustrates a cross section of the lead portion of the negative electrode, and 8 negative electrode current collectors 504 are bonded to each other by ultrasonic welding. For example, with an ultrasonic welder, a plurality of electrode layers are subjected to ultrasonic welding so as to be electrically connected to one another. The method of electrically connecting the current collectors is not limited to ultrasonic welding, and bolting may be employed. It is needless to say that the number of electrode layers is not limited to 16, and may be more than 16 or less than 16. In the case of a large number of electrode layers, the storage battery can have high capacity. In contrast, in the case of a small number of electrode layers, the storage battery can have small thickness and high flexibility.

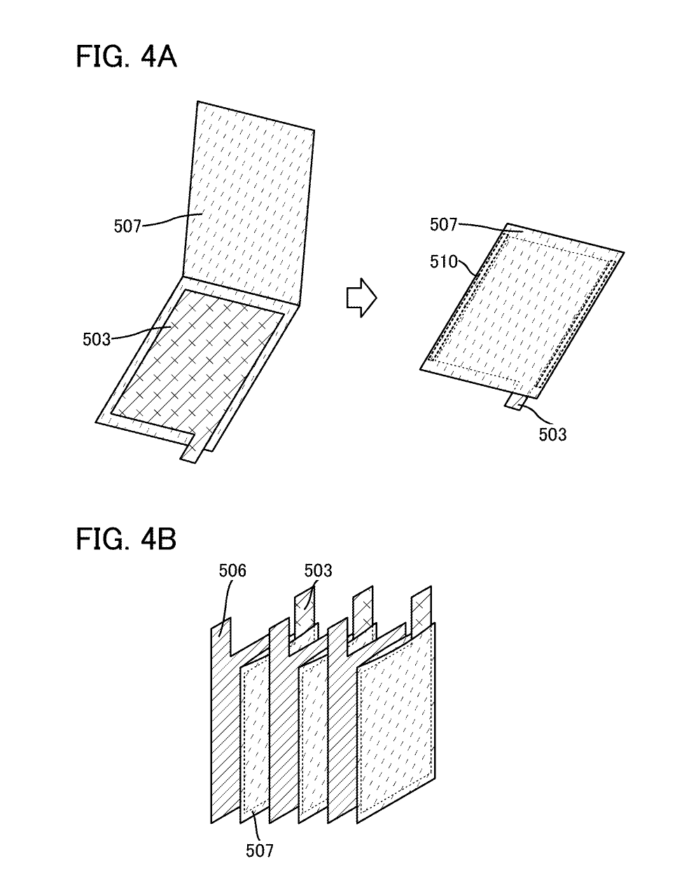

The separator 507 is preferably processed into a bag-like shape to surround one of the positive electrode 503 and the negative electrode 506. For example, as illustrated in FIG. 4A, the separator 507 is folded in two so that the positive electrode 503 is sandwiched, and sealed with a sealing member 510 in a region outside the region overlapping with the positive electrode 503; thus, the positive electrode 503 can be surely supported inside the separator 507. Then, as illustrated in FIG. 4B, the positive electrodes 503 which is surrounded by the separator 507 and the negative electrodes 506 are alternately stacked and provided in the exterior body 509; thus, the thin storage battery 500 can be formed.

Note that in this embodiment, the coin-type storage battery, the thin storage battery, and the cylindrical storage battery are given as examples of the storage battery; however, any of storage batteries with a variety of shapes, such as a sealed storage battery and a square-type storage battery, can be used. Furthermore, a structure in which a plurality of positive electrodes, a plurality of negative electrodes, and a plurality of separators are stacked or wound may be employed.

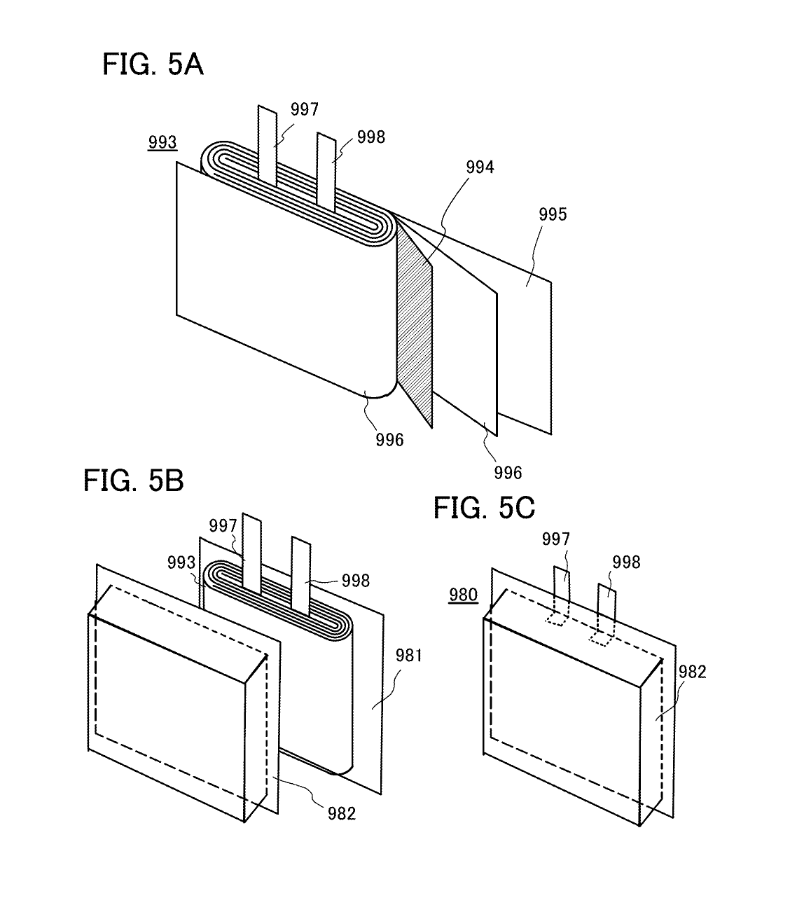

The thin storage battery is not limited to that illustrated in FIGS. 3A and 3B, and other examples of laminated storage batteries are illustrated in FIGS. 5A to 5C. A wound body 993 illustrated in FIG. 5A includes a negative electrode 994, a positive electrode 995, and a separator 996.

The wound body 993 is obtained by winding a sheet of a stack in which the negative electrode 994 overlaps with the positive electrode 995 with the separator 996 provided therebetween. The wound body 993 is covered with a rectangular sealed container or the like; thus, a rectangular secondary battery is fabricated.

Note that the number of stacks each including the negative electrode 994, the positive electrode 995, and the separator 996 may be determined as appropriate depending on capacity and an element volume which are required. The negative electrode 994 is connected to a negative electrode current collector (not illustrated) via one of a lead electrode 997 and a lead electrode 998. The positive electrode 995 is connected to a positive electrode current collector (not illustrated) via the other of the lead electrode 997 and the lead electrode 998.

In a power storage device 980 illustrated in FIGS. 5B and 5C, the wound body 993 is housed in a space formed by bonding a film 981 and a film 982 having a depressed portion by thermocompression bonding or the like. The wound body 993 includes the lead electrode 997 and the lead electrode 998, and is soaked in an electrolyte solution inside the film 981 and the film 982 having a depressed portion.

For the film 981 and the film 982 having a depressed portion, a metal material such as aluminum or a resin material can be used, for example. With the use of a resin material for the film 981 and the film 982 having a depressed portion, the film 981 and the film 982 having a depressed portion can be deformed when external force is applied; thus, a flexible storage battery can be manufactured. In the case where the film 981 and the film 982 having a depressed portion are deformed when external force is applied, adhesion between the current collector and the active material layer in contact with the current collector can be high by alloying part of the current collector.

The depressions of the film are formed by pressing, e.g., embossing. The depressions of a surface (or a rear surface) of the film that is formed by embossing form an obstructed space sealed by the film serving as a part of a wall of the sealing structure and whose inner volume is variable. This obstructed space can be said to be formed because the depressions of the film have an accordion structure (bellows structure). Note that embossing, which is a kind of pressing, is not necessarily employed and any method that allows formation of a relief on part of the film is employed.

Although FIGS. 5B and 5C illustrate an example where a space is formed by two films, the wound body 993 may be housed in a space formed by bending one film.

Furthermore, a flexible power storage device in which not only a thin storage battery has flexibility but also an exterior body and a sealed container have flexibility can be manufactured when a resin material or the like is used for the exterior body and the sealed container. Note that in the case where a resin material is used for the exterior body and the sealed container, a conductive material is used for a portion connected to the outside.

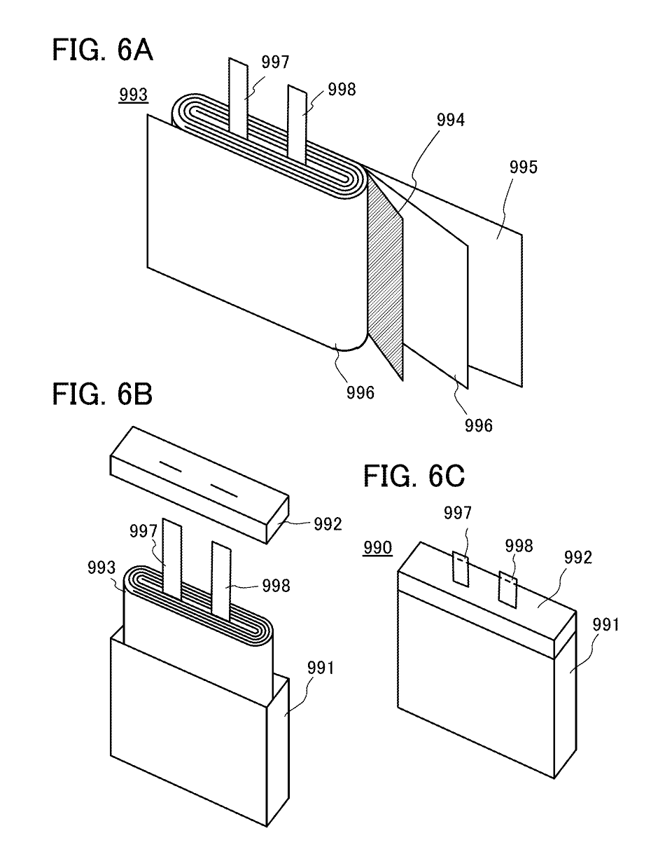

For example, FIGS. 6A to 6C illustrate an example of a flexible rectangular storage battery. The wound body 993 illustrated in FIG. 6A is the same as that illustrated in FIG. 5A, and a detailed description thereof is omitted.

In the power storage device 990 illustrated in FIGS. 6B and 6C, the wound body 993 is housed in an exterior body 991. The wound body 993 includes the lead electrode 997 and the lead electrode 998, and is soaked in an electrolyte solution inside the exterior body 991 and an exterior body 992. For example, a metal material such as aluminum or a resin material can be used for the exterior bodies 991 and 992. With the use of a resin material for the exterior bodies 991 and 992, the exterior bodies 991 and 992 can be deformed when external force is applied; thus, a flexible rectangular storage battery can be manufactured. In the case where the exterior bodies 991 and 992 are deformed when external force is applied, adhesion between the current collector and the active material layer in contact with the current collector can be high by alloying part of the current collector.

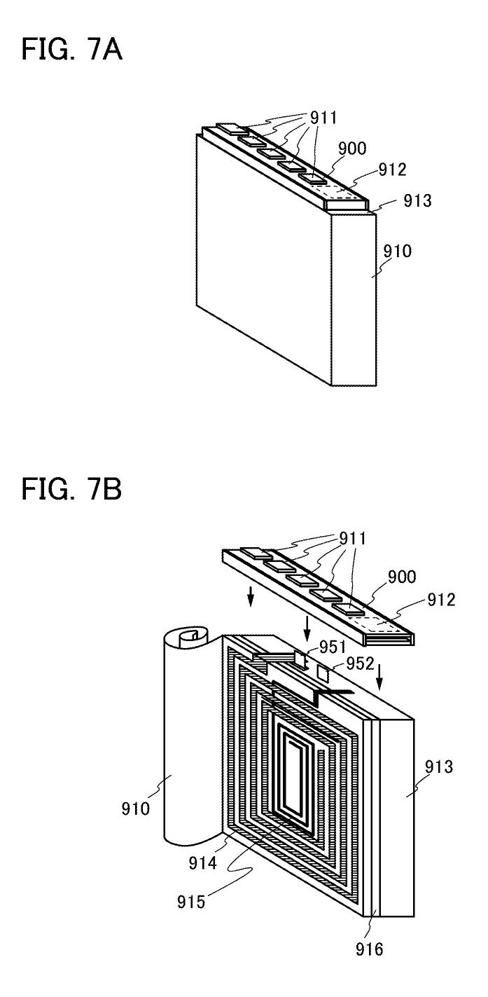

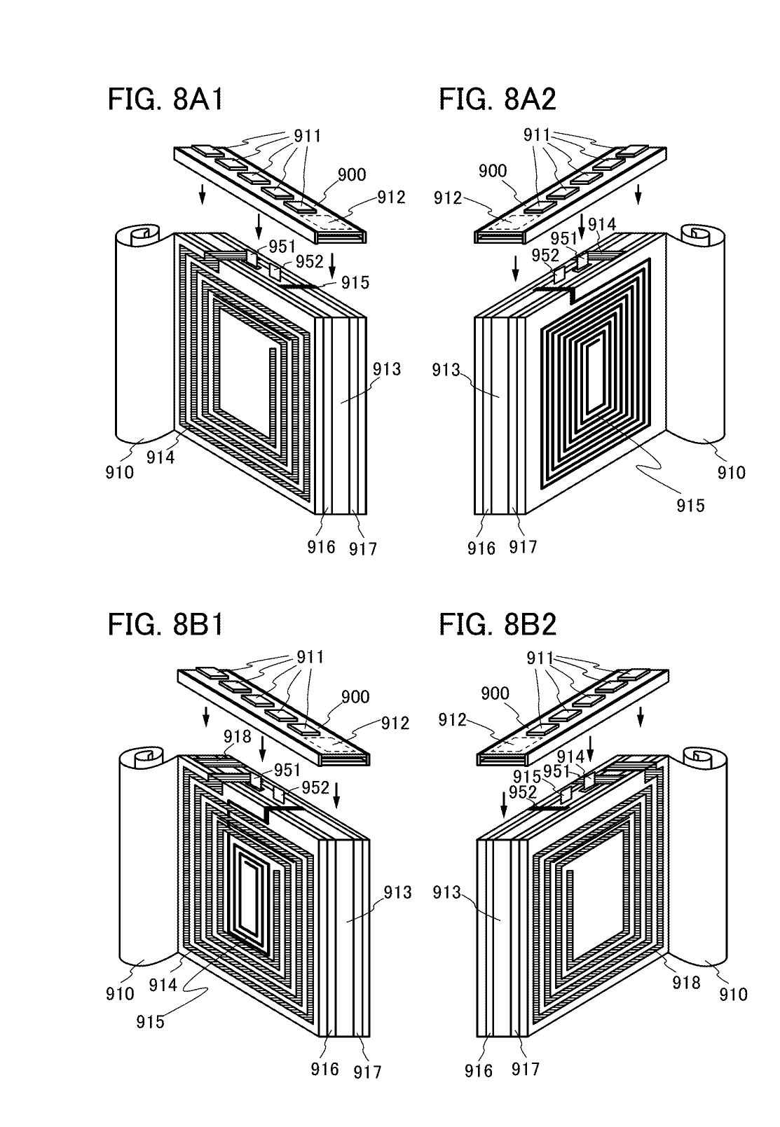

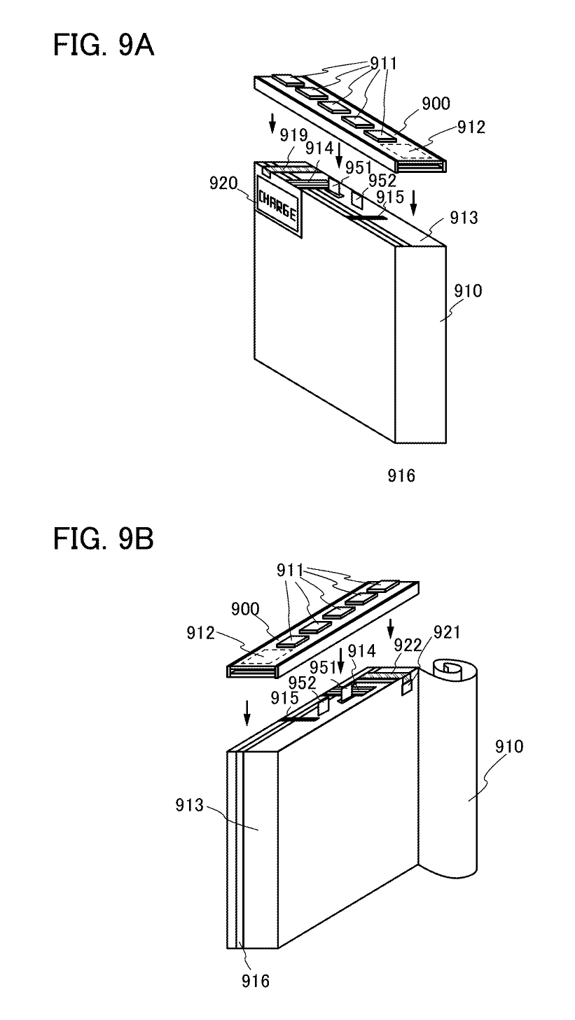

A structural example of a power storage device (power storage unit) is described with reference to FIGS. 7A and 7B, FIGS. 8A1, 8A2, 8B1, and 8B2, and FIGS. 9A and 9B.

FIGS. 7A and 7B are external views of a power storage device. The power storage device includes a circuit board 900 and a power storage unit 913. A label 910 is attached to the power storage unit 913. As shown in FIG. 7B, the power storage device further includes a terminal 951 and a terminal 952, and includes an antenna 914 and an antenna 915 between the power storage unit 913 and the label 910.

The circuit board 900 includes terminals 911 and a circuit 912. The terminals 911 are connected to the terminals 951 and 952, the antennas 914 and 915, and the circuit 912. Note that a plurality of terminals 911 serving as a control signal input terminal, a power supply terminal, and the like may be provided.

The circuit 912 may be provided on the rear surface of the circuit board 900. The shape of each of the antennas 914 and 915 is not limited to a coil shape and may be a linear shape or a plate shape. Furthermore, a planar antenna, an aperture antenna, a traveling-wave antenna, an EH antenna, a magnetic-field antenna, or a dielectric antenna may be used. Alternatively, the antenna 914 or the antenna 915 may be a flat-plate conductor. The flat-plate conductor can serve as one of conductors for electric field coupling. That is, the antenna 914 or the antenna 915 can serve as one of two conductors of a capacitor. Thus, electric power can be transmitted and received not only by an electromagnetic field or a magnetic field but also by an electric field.

The line width of the antenna 914 is preferably larger than that of the antenna 915. This makes it possible to increase the amount of electric power received by the antenna 914.

The power storage device includes a layer 916 between the power storage unit 913 and the antennas 914 and 915. The layer 916 has a function of blocking an electromagnetic field from the power storage unit 913, for example. As the layer 916, for example, a magnetic body can be used.

Note that the structure of the power storage device is not limited to that illustrated in FIGS. 7A and 7B.

For example, as illustrated in FIGS. 8A1 and 8A2, two opposite surfaces of the power storage unit 913 in FIGS. 7A and 7B may be provided with respective antennas. FIG. 8A1 is an external view illustrating one side of the opposing surfaces, and FIG. 8A2 is an external view illustrating the other side of the opposing surfaces. Note that description on the power storage device shown in FIGS. 7A and 7B can be referred to for portions similar to those in FIGS. 7A and 7B, as appropriate.

As illustrated in FIG. 8A1, the antenna 914 is provided on one of the opposing surfaces of the power storage unit 913 with the layer 916 provided therebetween, and as illustrated in FIG. 8A2, an antenna 915 is provided on the other of the opposing surfaces of the power storage unit 913 with the layer 917 provided therebetween. The layer 917 has a function of blocking an electromagnetic field from the power storage unit 913. As the layer 917, for example, a magnetic body can be used. The layer 917 may serve as a shielding layer.

With the above structure, both the antenna 914 and the antenna 915 can be increased in size.

Alternatively, as illustrated in FIGS. 8B1 and 8B2, two opposite surfaces of the power storage unit 913 in FIGS. 7A and 7B may be provided with different types of antennas. FIG. 8B1 is an external view illustrating one of the opposite surfaces, and FIG. 8B2 is an external view illustrating the other of the opposite surfaces. Note that description on the power storage device shown in FIGS. 7A and 7B can be referred to for portions similar to those in FIGS. 7A and 7B, as appropriate.

As illustrated in FIG. 8B1, the antennas 914 and 915 are provided on one of the opposite surfaces of the power storage unit 913 with the layer 916 provided therebetween, and as illustrated in FIG. 8A2, an antenna 918 is provided on the other of the opposite surfaces of the power storage unit 913 with the layer 917 provided therebetween. The antenna 918 has a function of performing data communication with an external device, for example. An antenna with a shape that can be applied to the antennas 914 and 915, for example, can be used as the antenna 918. As a system for communication using the antenna 918 between the power storage device and an external device, a response method that can be used between the power storage device and the external device, such as NFC, can be employed.

Alternatively, as illustrated in FIG. 9A, the power storage unit 913 in FIGS. 7A and 7B may be provided with a display device 920. The display device 920 is electrically connected to the terminal 911 via a terminal 919. It is possible that the label 910 is not provided in a portion where the display device 920 is provided. Note that description on the power storage device shown in FIGS. 7A and 7B can be referred to for portions similar to those in FIGS. 7A and 7B, as appropriate.

The display device 920 can display, for example, an image showing whether or not charging is being carried out or an image showing the amount of stored power. As the display device 920, electronic paper, a liquid crystal display device, an electroluminescent (EL) display device, or the like can be used. For example, power consumption of the display device 920 can be reduced when electronic paper is used.

Alternatively, as illustrated in FIG. 9B, the power storage unit 913 in FIGS. 7A and 7B may be provided with a sensor 921. The sensor 921 is electrically connected to the terminal 911 via a terminal 922. Note that the sensor 921 may be provided between the power storage unit 913 and the label 910. Note that description on the power storage device shown in FIGS. 7A and 7B can be referred to for portions similar to those in FIGS. 7A and 7B, as appropriate.

As the sensor 921, a sensor having a function of measuring force, displacement, position, speed, acceleration, angular velocity, rotational frequency, distance, light, liquid, magnetism, temperature, chemical substance, sound, time, hardness, electric field, electric current, voltage, electric power, radiation, flow rate, humidity, gradient, oscillation, odor, or infrared rays can be used. With the sensor 921, for example, data on an environment (e.g., temperature) where the power storage device is placed can be detected and stored in a memory inside the circuit 912.

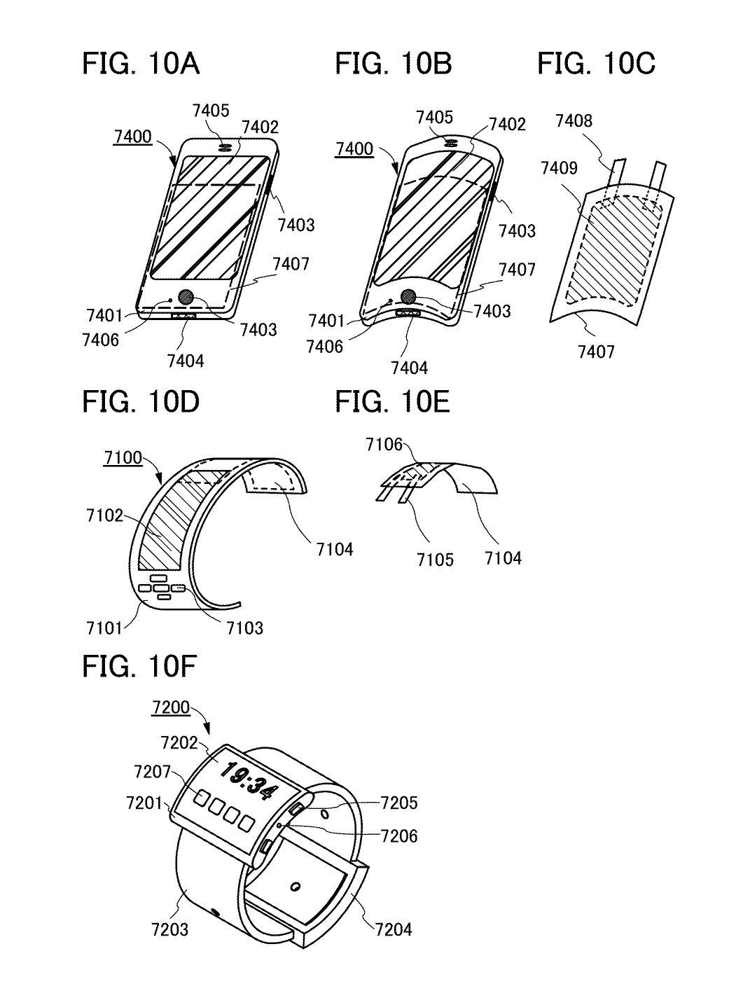

FIGS. 10A to 10E illustrate examples of electronic devices including flexible storage batteries illustrated in FIGS. 3A and 3B, FIGS. 5A to 5C, and FIGS. 6A to 6C. Examples of an electronic device including a flexible power storage device include television devices (also referred to as televisions or television receivers), monitors of computers or the like, cameras such as digital cameras or digital video cameras, digital photo frames, mobile phones (also referred to as mobile phones or mobile phone devices), portable game machines, portable information terminals, audio reproducing devices, large game machines such as pachinko machines, and the like.

In addition, a flexible power storage device can be incorporated along a curved inside/outside wall surface of a house or a building or a curved interior/exterior surface of a car.

FIG. 10A illustrates an example of a mobile phone. A mobile phone 7400 is provided with a display portion 7402 incorporated in a housing 7401, an operation button 7403, an external connection port 7404, a speaker 7405, a microphone 7406, and the like. Note that the mobile phone 7400 includes a power storage device 7407.

The mobile phone 7400 illustrated in FIG. 10B is bent. When the whole mobile phone 7400 is bent by the external force, the power storage device 7407 included in the mobile phone 7400 is also bent. FIG. 10C illustrates the bent power storage device 7407. The power storage device 7407 is a thin storage battery. The power storage device 7407 is fixed in a state of being bent. Note that the power storage device 7407 includes a lead electrode 7408 electrically connected to a current collector 7409. The current collector 7409 is, for example, a metal foil containing copper as its main component, and partly alloyed with gallium; thus, adhesion between the current collector 7409 and an active material layer in contact with the current collector 7409 is improved and the power storage device 7407 can have high reliability even in a state of being bent.

FIG. 10D illustrates an example of a bangle display device. A portable display device 7100 includes a housing 7101, a display portion 7102, an operation button 7103, and a power storage device 7104. FIG. 10E illustrates the bent power storage device 7104. When a user wears the power storage device 7104 in a state of being bent on the wrist, a housing of the power storage device 7104 is deformed and the curvature thereof is partly or entirely changed. Note that the radius of curvature of a curve at a point is a measure of the radius of the circular arc which best approximates the curve at that point. The reciprocal of radius of curvature is referred to as curvature. Specifically, a main surface of the housing or a main surface of the power storage device 7104 partly or totally changes to have a radius of curvature R of greater than or equal to 40 mm and less than or equal to 150 mm. The radius of curvature R at the main surface of the power storage device 7104 is greater than or equal to 40 mm and less than or equal to 150 mm, the reliability can be kept high. Note that the power storage device 7104 includes a lead electrode 7105 electrically connected to a current collector 7106. The current collector 7106 is, for example, a metal foil containing copper as its main component, and partly alloyed with gallium; thus, adhesion between the current collector 7106 and an active material layer in contact with the current collector 7106 is improved and the power storage device 7104 can have high reliability even when the power storage device 7104 is bent and its curvature is changed many times.

FIG. 10F illustrates an example of a wrist-watch-type portable information terminal. A portable information terminal 7200 includes a housing 7201, a display portion 7202, a band 7203, a buckle 7204, an operation button 7205, an input output terminal 7206, and the like.

The portable information terminal 7200 is capable of executing a variety of applications such as mobile phone calls, e-mailing, viewing and editing texts, music reproduction, Internet communication, and a computer game.

The display surface of the display portion 7202 is bent, and images can be displayed on the bent display surface. Further, the display portion 7202 includes a touch sensor, and operation can be performed by touching the screen with a finger, a stylus, or the like. For example, by touching an icon 7207 displayed on the display portion 7202, application can be started.

With the operation button 7205, a variety of functions such as power ON/OFF, ON/OFF of wireless communication, setting and cancellation of manner mode, and setting and cancellation of power saving mode can be performed. For example, the functions of the operation button 7205 can be set freely by setting the operation system incorporated in the portable information terminal 7200.

Further, the portable information terminal 7200 can employ near field communication that is a communication method based on an existing communication standard. In that case, for example, mutual communication between the portable information terminal 7200 and a headset capable of wireless communication can be performed, and thus hands-free calling is possible.

Moreover, the portable information terminal 7200 includes the input output terminal 7206, and data can be directly transmitted to and received from another information terminal via a connector. Power charging through the input output terminal 7206 is possible. Note that the charging operation may be performed by wireless power feeding without using the input output terminal 7206.

The display portion 7202 of the portable information terminal 7200 includes the power storage device with an electrode member of one embodiment of the present invention. For example, the power storage device 7104 illustrated in FIG. 10E can be incorporated in the housing 7201 with a state where the power storage device 7104 is bent or can be incorporated in the band 7203 with a state where the power storage device 7104 can be bent.

The use of storage batteries in vehicles can lead to next-generation clean energy vehicles such as hybrid electric vehicles (HEVs), electric vehicles (EVs), and plug-in hybrid electric vehicles (PHEVs).

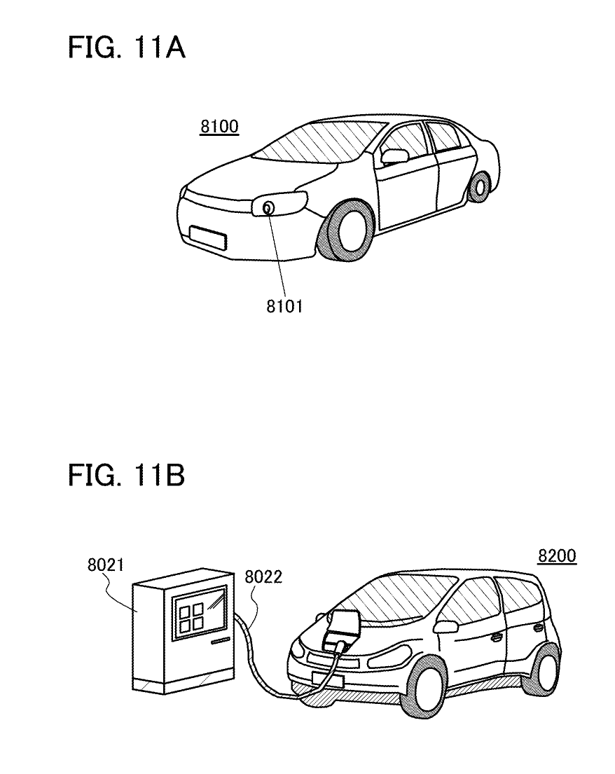

FIGS. 11A and 11B each illustrate an example of a vehicle using one embodiment of the present invention. An automobile 8100 illustrated in FIG. 11A is an electric vehicle which runs on the power of the electric motor. Alternatively, the automobile 8100 is a hybrid electric vehicle capable of driving using either the electric motor or the engine as appropriate. One embodiment of the present invention achieves a high-mileage vehicle. The automobile 8100 includes the power storage device. The power storage device is used not only for driving an electric motor, but also for supplying electric power to a light-emitting device such as a headlight 8101 or a room light (not illustrated).

The power storage device can also supply electric power to a display device included in the automobile 8100, such as a speedometer or a tachometer. Furthermore, the power storage device can supply electric power to a semiconductor device included in the automobile 8100, such as a navigation system.

FIG. 11B illustrates an automobile 8200 including the power storage device. The automobile 8200 can be charged when the power storage device is supplied with electric power through external charging equipment by a plug-in system, a contactless power supply system, or the like. In FIG. 11B, the power storage device included in the automobile 8200 is charged with the use of a ground-based charging apparatus 8021 through a cable 8022. In charging, a given method such as CHAdeMO® or Combined Charging System may be referred to for a charging method, the standard of a connector, or the like as appropriate. The charging apparatus 8021 may be a charging station provided in a commerce facility or a power source in a house. For example, with the use of a plug-in technique, a power storage device 8024 included in the automobile 8200 can be charged by being supplied with electric power from outside. The charging can be performed by converting AC electric power into DC electric power through a converter such as an AC-DC converter.

Further, although not illustrated, the vehicle may include a power receiving device so as to be charged by being supplied with electric power from an above-ground power transmitting device in a contactless manner. In the case of the contactless power supply system, by fitting the power transmitting device in a road or an exterior wall, charging can be performed not only when the automobile stops but also when moves. In addition, the contactless power supply system may be utilized to perform transmission/reception between two vehicles. Furthermore, a solar cell may be provided in the exterior of the automobile to charge the power storage device when the automobile stops or moves. To supply electric power in such a contactless manner, an electromagnetic induction method or a magnetic resonance method can be used.

According to one embodiment of the present invention, the power storage device can have improved cycle characteristics and reliability. Furthermore, according to one embodiment of the present invention, the power storage device itself can be made more compact and lightweight as a result of improved characteristics of the power storage device. The compact and lightweight power storage device contributes to a reduction in the weight of a vehicle, and thus increases the driving distance. Further, the power storage device included in the vehicle can be used as a power source for supplying electric power to products other than the vehicle. In that case, the use of a commercial power supply can be avoided at peak time of electric power demand.

At least part of this embodiment can be implemented in combination with any of the other embodiments described in this specification as appropriate.

### Example 1

In this example, described is an example of synthesizing 1-methyl-3-(2-propoxyethyl)imidazolium bis(fluorosulfonyl)amide (abbreviation: poEMI-FSA) that can be used for a nonaqueous solvent in a nonaqueous electrolyte used for a power storage device of one embodiment of the present invention, and that is represented by the following structural formula.

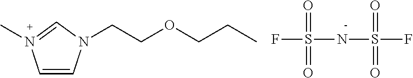

### Synthesis of 1-methyl-3-(2-propoxyethyl)imidazolium chloride

Into a 100-mL three-neck flask were put 8.27 g (101 mmol) of 1-methylimidazole, 13.4 g (109 mmol) of 2-chloroethylpropyl ether, and 5 mL acetonitrile. This solution was stirred at 80° C. in a nitrogen stream for six hours and at 100° C. for eight hours. After the reaction, ethyl acetate was added to the solution and the obtained solution was further stirred. The organic layer was removed to wash the solution. To the obtained aqueous layer were added 100 mL of acetonitrile and 5.27 g of activated carbon, and the solution was stirred for 20 hours. After the stirring, the aqueous layer was subjected to suction filtration through Celite (produced by Wako Pure Chemical Industries, Ltd., Catalog No. 537-02305), and the obtained filtrate was concentrated. Water was added to the obtained solution, and an aqueous layer was washed with ethyl acetate. This aqueous layer was concentrated and dried, so that 17.0 g of the target yellow liquid was obtained in a yield of 82%.

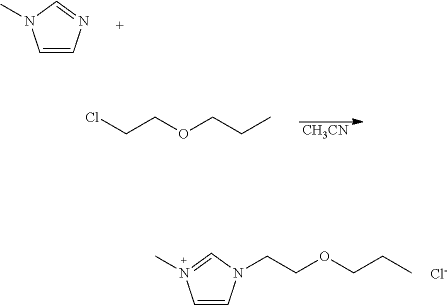

By a nuclear magnetic resonance (NMR) method, the compound synthesized through the above steps was identified as the target 1-methyl-3-(2-propoxyethyl)imidazolium chloride.

¹H NMR data of the obtained compound is shown below. ¹H NMR (CDCl₃, 300 MHz):δ=0.88 (t, J=7.2 Hz, 3H), 1.51-1.63 (m, 2H), 3.42 (t, J=6.9 Hz, 2H), 3.79-3.82 (m, 2H), 4.08 (s, 3H), 4.59-4.62 (m, 2H), 7.21-7.22 (m, 1H), 7.43-7.44 (m, 1H), 10.70 (s, 1H).

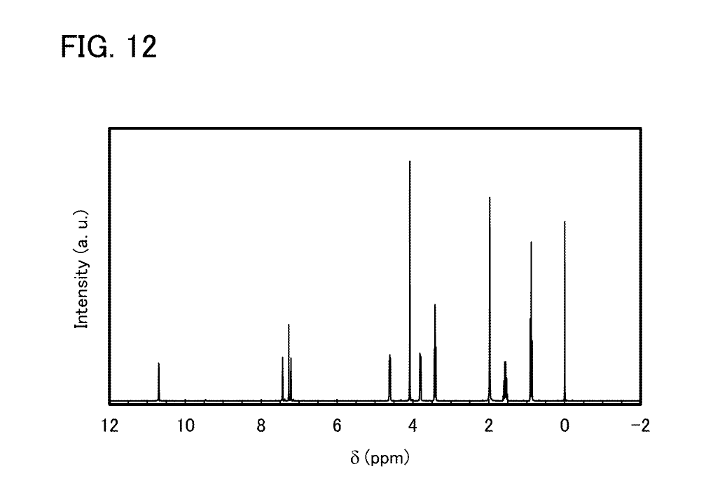

FIG. 12 is a ¹H NMR chart.

<Synthesis of poEMI-FSA>

Into a 100-mL recovery flask were put 17.0 g (83.1 mmol) of 1-methyl-3-(2-propoxyethyl)imidazolium chloride, 20.1 g (91.7 mmol) of potassium bis(fluorosulfonyl)amide, and 20 mL of water. The resulting solution was stirred for 20 hours at room temperature. After the reaction, water was added to the obtained solution, and an aqueous layer was subjected to extraction with dichloromethane. The extracted solution and an organic layer were combined and the mixture was washed with water, and then, the organic layer was dried with magnesium sulfate. The solution was gravity-filtered to remove the magnesium sulfate, and the obtained filtrate was concentrated and dried, so that 26.2 g of the target yellow liquid was obtained in a yield of 90%.

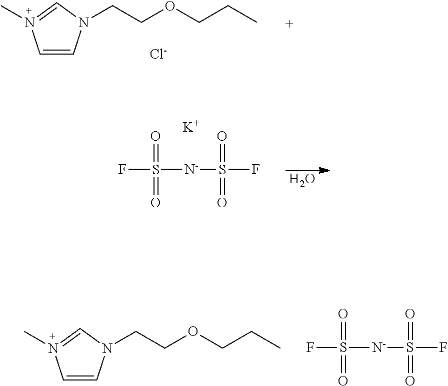

The compound synthesized through the above steps was identified as poEMI-FSA, which was the target compound, by a nuclear magnetic resonance (NMR).

¹H NMR data of the obtained compound is shown below. ¹H NMR (1,1,2,2-tetrachloroethane-d₂, 300 MHz):δ=0.90 (t, J=7.5 Hz, 3H), 1.53-1.65 (m, 2H), 3.44 (t, J=6.9 Hz, 2H), 3.74-3.77 (m, 2H), 3.96 (s, 3H), 4.33-4.36 (m, 2H), 7.22-7.23 (m, 1H), 7.40-7.41 (m, 1H), 8.58 (s, 1H).

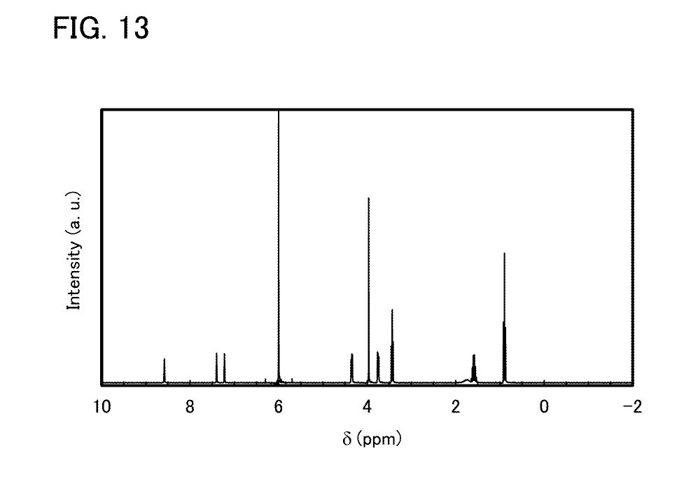

FIG. 13 is a ¹H NMR chart.

The results indicate that poEMI-FSA was synthesized.

### Example 2

In this example, described is an example of synthesizing 1-(4-methoxybutyl)-3-methylimidazolium bis(fluorosulfonyl)amide (abbreviation: moBMI-FSA) that can be used for a nonaqueous solvent in a nonaqueous electrolyte used for a power storage device of one embodiment of the present invention, and that is represented by the following structural formula.

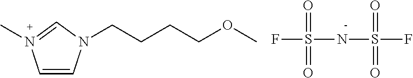

### Synthesis of 1-(4-methoxybutyl)-3-methylimidazolium chloride

Into a 100-mL three-neck flask was put 8.28 g (101 mmol) of 1-methylimidazole, and the temperature was lowered to 0° C. in a nitrogen stream and 12.6 g (103 mmol) of 1-chloro-4-methoxybutane was added thereto. This solution was stirred at 80° C. for seven hours. After the reaction, ethyl acetate was added to the solution and the obtained solution was further stirred. The organic layer was removed to wash the solution. To the obtained aqueous layer were added 100 mL of acetonitrile and 6.74 g of activated carbon, and the solution was stirred for 20 hours. After the stirring, the aqueous layer was subjected to suction filtration through Celite (produced by Wako Pure Chemical Industries, Ltd., Catalog No. 537-02305), and the obtained filtrate was concentrated. Water was added to the obtained solution, and an aqueous layer was washed with ethyl acetate. This aqueous layer was concentrated and dried, so that 12.5 g of the target light yellow liquid was obtained in a yield of 60%.

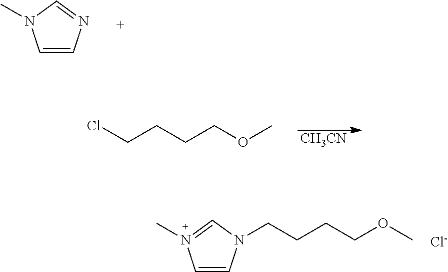

By a nuclear magnetic resonance (NMR) method, the compound synthesized through the above steps was identified as the target 1-(4-methoxybutyl)-3-methylimidazolium chloride.

¹H NMR data of the obtained compound is shown below. ¹H NMR (DMSO-d₆, 300 MHz):δ=1.43-1.52 (m, 2H), 1.78-1.88 (m, 2H), 3.22 (s, 3H), 3.31-3.35 (m, 2H), 3.85 (s, 3H), 4.17 (t, J=7.2 Hz, 2H), 7.70-7.71 (m, 1H), 7.77-7.78 (m, 1H), 9.13 (s, 1H).

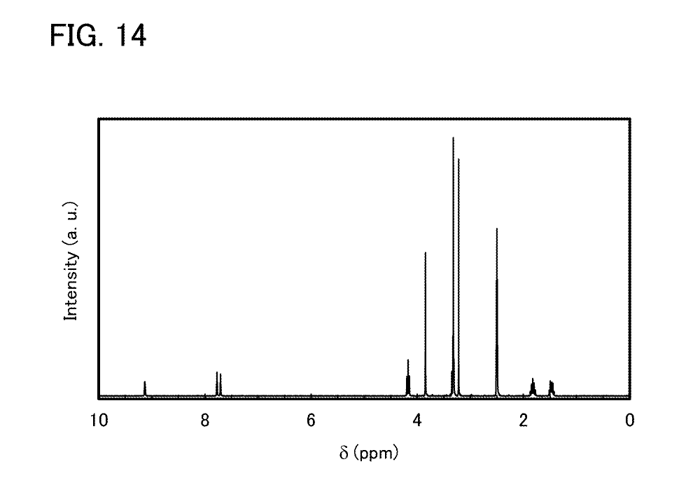

FIG. 14 is a ¹H NMR chart.

<Synthesis of moBMI-FSA>

Into a 200-mL recovery flask were put 12.5 g (61.2 mmol) of 1-(4-methoxybutyl)-3-methylimidazolium chloride, 13.9 g (63.2 mmol) of potassium bis(fluorosulfonyl)amide, and 30 mL of water. The resulting solution was stirred for 91 hours at room temperature. After the reaction, water was added to the obtained solution, and an aqueous layer was subjected to extraction with dichloromethane. The extracted solution and an organic layer were combined and the mixture was washed with water, and then, the organic layer was dried with magnesium sulfate. The solution was gravity-filtered to remove the magnesium sulfate, and the obtained filtrate was concentrated and dried, so that 17.9 g of the target transparent liquid was obtained in a yield of 83%.

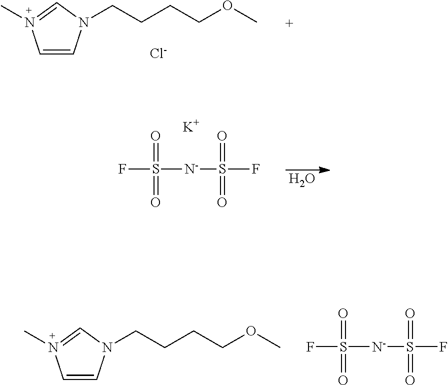

The compound synthesized through the above steps was identified as moBMI-FSA, which was the target compound, by a nuclear magnetic resonance (NMR).

¹H NMR data of the obtained compound is shown below. ¹H NMR (CDCl₃, 300 MHz):δ=1.56-1.65 (m, 2H), 1.98 (quin, J=7.5 Hz, 2H), 3.32 (s, 3H), 3.43 (t, J=6.0 Hz, 2H), 3.95 (s, 3H), 4.24 (t, J=7.5 Hz, 2H), 7.30-7.34 (m, 2H), 8.62 (s, 1H).

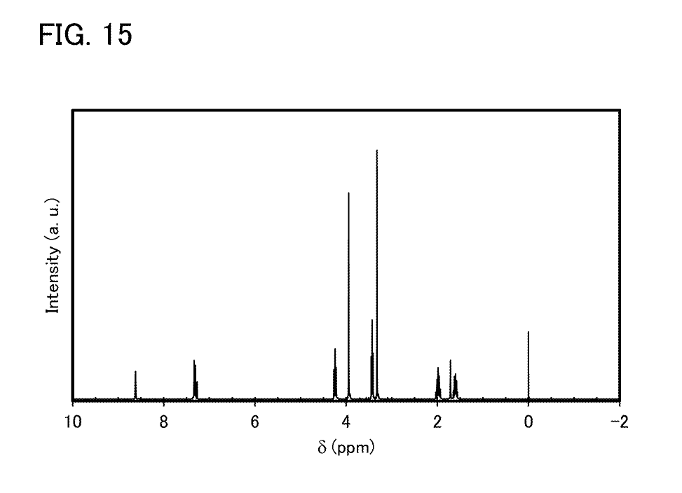

FIG. 15 is a ¹H NMR chart.

The results indicate that moBMI-FSA was synthesized.

### Example 3

In this example, described is an example of synthesizing 1-hexyl-3-methylimidazolium bis(fluorosulfonyl)amide (abbreviation: HMI-FSA) that can be used for a nonaqueous solvent in a nonaqueous electrolyte used for a power storage device of one embodiment of the present invention, and that is represented by the following structural formula.

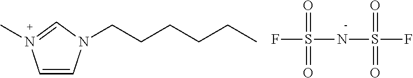

### <Synthesis of HMI-FSA>

Into a 200-mL conical flask were put 22.7 g (91.9 mmol) of 1-hexyl-3-methylimidazolium bromide, 22.1 g (101 mmol) of potassium bis(fluorosulfonyl)amide, and 40 mL of water. The resulting solution was stirred for 19 hours at room temperature. After the reaction, the obtained solution was subjected to extraction with dichloromethane. The extracted solution and an organic layer were combined and the mixture was washed with water, and then, the organic layer was dried with magnesium sulfate. The solution was gravity-filtered to remove the magnesium sulfate, and the obtained filtrate was concentrated and dried, so that 28.6 g of the target yellow liquid was obtained in a yield of 89%.

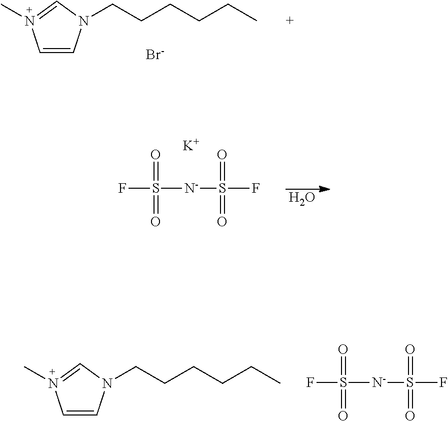

By a nuclear magnetic resonance (NMR) method, the compound synthesized through the above steps was identified as the target HMI-FSA.

¹H NMR data of the obtained compound is shown below. ¹H NMR (CDCl₃, 300 MHz):δ=0.86-0.91 (m, 3H), 1.33-1.37 (m, 6H), 1.83-1.91 (m, 2H), 3.96 (s, 3H), 4.18 (t, J=7.8 Hz, 2H), 7.27-7.30 (m, 2H), 8.66 (s, 1H).

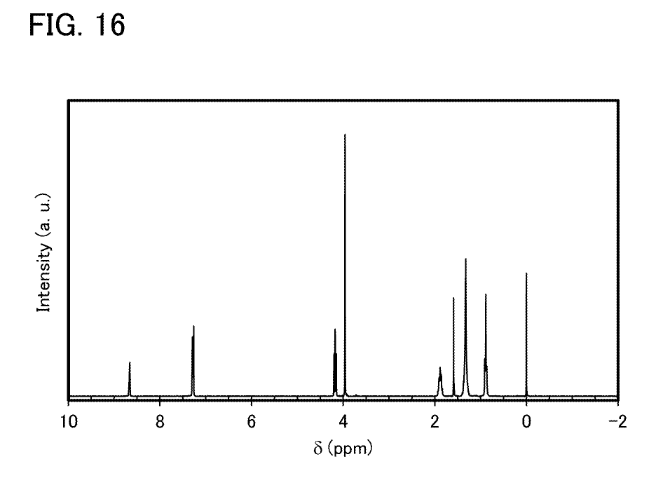

FIG. 16 is a ¹H NMR chart.

The results indicate that HMI-FSA was synthesized.

### Example 4

In this example, described is an example of synthesizing 3-[(2-methoxyethoxymethyl)]-1-methylimidazo lium bis(fluorosulfonyl)amide (abbreviation: meoM2I-FSA) that can be used for a nonaqueous solvent in a nonaqueous electrolyte used for a power storage device of one embodiment of the present invention, and that is represented by the following structural formula.

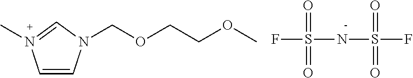

### Synthesis of 3-(2-methoxyethoxymethyl)-1-methylimidazolium chloride

Into a 100-mL three-neck flask were put 8.23 g (100 mmol) of 1-methylimidazole and 5 mL of acetonitrile. This solution was cooled to 0° C. in a nitrogen stream, and 12.5 g (100 mmol) of 2-methoxyethoxymethyl chloride was dropped thereto. After the dropping, the temperature of this solution was raised to room temperature and stirring was performed for four days. After the reaction, ethyl acetate was added to the obtained solution and the solution was stirred. The organic layer was removed to wash the solution. To the obtained aqueous layer were added 100 mL of acetonitrile and 6.47 g of activated carbon, and stirring was performed for 24 hours. After the stirring, the aqueous layer was subjected to suction filtration through Celite, and the obtained filtrate was concentrated. Water was added to the obtained solution and an aqueous layer was washed with ethyl acetate. This aqueous layer was concentrated and dried, 16.4 g of the target transparent liquid was obtained in a yield of 79%.

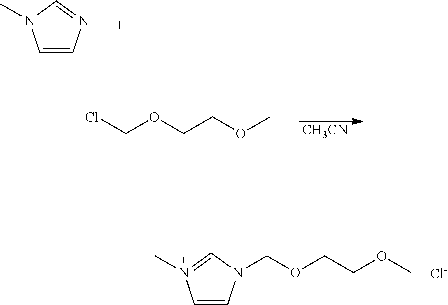

By a nuclear magnetic resonance (NMR) method, the compound synthesized through the above steps was identified as the target 3-(2-methoxyethoxymethyl)-1-methylimidazolium chloride.

¹H NMR data of the obtained compound is shown below. ¹H NMR (CDCl₃, 300 MHz):δ=3.34 (s, 3H), 3.54-3.56 (m, 2H), 3.85-3.88 (m, 2H), 4.11 (s, 3H), 5.88 (s, 2H), 7.25-7.26 (m, 1H), 7.45-7.47 (m, 1H), 11.20 (s, 1H).

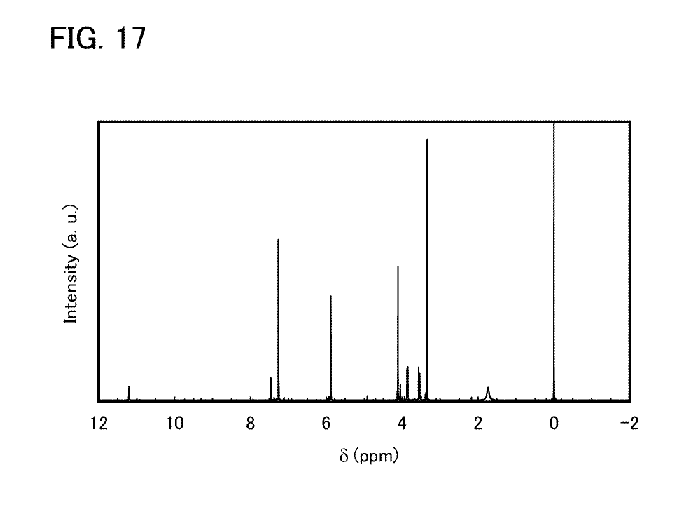

FIG. 17 is a ¹H NMR chart.

<Synthesis of meoM2I-FSA>

Into a 200-mL recovery flask were put 16.4 g (79.3 mmol) of 3-(2-methoxyethoxymethyl)-3-methylimidazolium chloride, 19.2 g (87.4 mmol) of potassium bis(fluorosulfonyl)amide, and 30 mL of water. The resulting solution was stirred for 17 hours at room temperature. After the reaction, water was added to the obtained solution, and an aqueous layer was subjected to extraction with dichloromethane. The extracted solution and an organic layer were combined and the mixture was washed with water, and then, the organic layer was dried with magnesium sulfate. The solution was gravity-filtered to remove the magnesium sulfate, and the obtained filtrate was concentrated and dried, so that 21.2 g of the target transparent liquid was obtained in a yield of 76%.

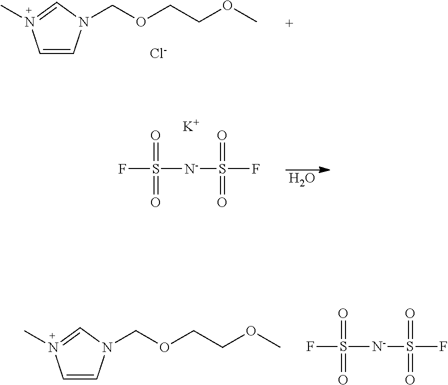

The compound synthesized through the above steps was identified as meoM2I-FSA, which was the target compound, by a nuclear magnetic resonance (NMR).

¹H NMR data of the obtained compound is shown below. ¹H NMR (DMSO-d₆, 300 MHz):δ=3.22 (s, 3H), 3.43-3.46 (m, 2H), 3.63-3.66 (m, 2H), 3.89 (s, 3H), 5.57 (s, 2H), 7.75-7.76 (m, 1H), 7.83-7.84 (m, 1H), 9.26 (s, 1H).

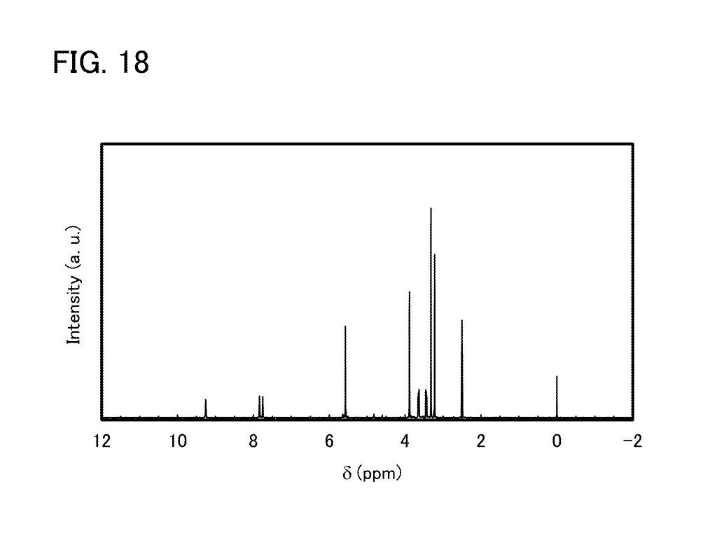

FIG. 18 is a ¹H NMR chart.

The results indicate that meoM2I-FSA was synthesized.

### Example 5

In this example, described is an example of synthesizing 3-[2-(methoxymethoxy)ethyl]-1-methylimidazolium bis(fluorosulfonyl)amide (abbreviation: mo2EMI-FSA) that can be used for a nonaqueous solvent in a nonaqueous electrolyte used for a power storage device of one embodiment of the present invention, and that is represented by the following structural formula.

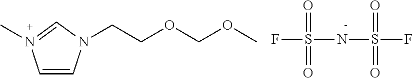

### Synthesis of 3-[2-(methoxymethoxy)ethyl]-1-methylimidazolium bromide

Into a 100-mL three-neck flask were put 7.25 g (88.3 mmol) of 1-methylimidazole, 5 mL of acetonitrile, and 10.2 g (60.4 mmol) of 1-bromo-2-(methoxymethoxy)ethane. This solution was stirred at 80° C. in a nitrogen stream for seven hours. After the reaction, ethyl acetate was added to the solution and the obtained solution was further stirred. The organic layer was removed to wash the solution. To the obtained aqueous layer were added 100 mL of acetonitrile and 6.69 g of activated carbon, and the solution was stirred for three days. The mixture was subjected to suction filtration through Celite, and the obtained filtrate was concentrated Water was added to the obtained solution, and an aqueous layer was washed with ethyl acetate. This aqueous layer was concentrated and dried, so that 13.2 g of the target transparent liquid was obtained in a yield of 87%.

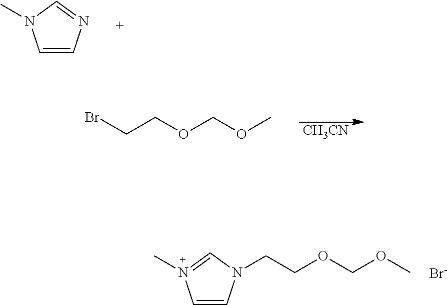

By a nuclear magnetic resonance (NMR) method, the compound synthesized through the above steps was identified as the target 3-[2-(methoxymethoxy)ethyl]-1-methyl-1-imidazolium bromide.

¹H NMR data of the obtained compound is shown below. ¹H NMR (CDCl₃, 300 MHz):δ=3.32 (s, 3H), 3.93-3.96 (m, 2H), 4.09 (s, 3H), 4.63-4.66 (m, 4H), 7.16 (s, 1H), 7.40 (s, 1H), 10.75 (s, 1H).

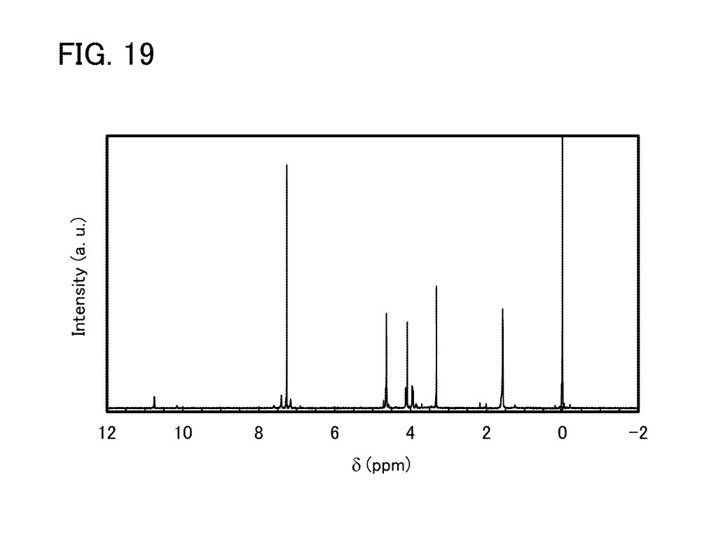

FIG. 19 is a ¹H NMR chart.

<Synthesis of mo2EMI-FSA>

Into a 200-mL recovery flask were put 13.2 g (52.7 mmol) of 3-[2-(methoxymethoxy)ethyl]-1-methylimidazolium bromide, 12.8 g (58.3 mmol) of potassium bis(fluorosulfonyl)amide, and 30 mL of water. The resulting solution was stirred for 17 hours at room temperature. After the reaction, water was added to the obtained solution, and an aqueous layer was subjected to extraction with dichloromethane. The extracted solution and an organic layer were combined and the mixture was washed with water, and then, the organic layer was dried with magnesium sulfate. The solution was gravity-filtered to remove the magnesium sulfate, and the obtained filtrate was concentrated and dried, so that 15.0 g of the target transparent liquid was obtained in a yield of 81%.

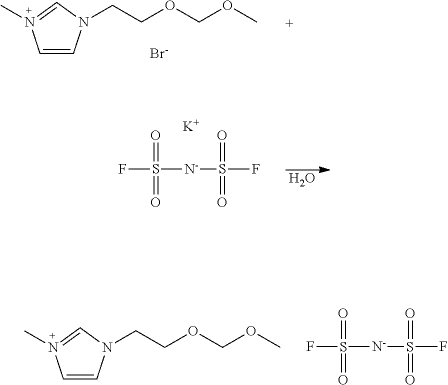

The compound synthesized through the above steps was identified as mo2EMI-FSA, which was the target compound, by a nuclear magnetic resonance (NMR).

¹H NMR data of the obtained compound is shown below. ¹H NMR (CDCl₃, 300 MHz):δ=3.33(s, 3H), 3.89-3.92 (m, 2H), 4.00 (s, 3H), 4.41-4.45 (m, 2H), 4.64 (s, 2H), 7.22 (s, 1H), 7.40 (s, 1H), 8.82 (s, 1H).

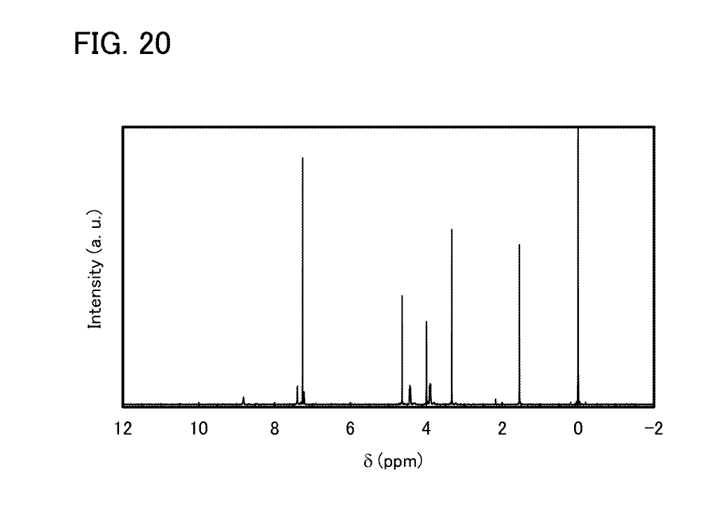

FIG. 20 is a ¹H NMR chart.

The results indicate that mo2EMI-FSA was synthesized.

### Example 6

In this example, power storage devices were fabricated with the use of the nonaqueous electrolyte described in the above embodiment, and the power storage devices were evaluated. As the power storage devices, coin-type lithium ion secondary batteries were fabricated. The coin-type lithium ion secondary battery in this example has a lithium iron phosphate-graphite full-cell structure, in which lithium iron phosphate (LiFePO₄) was used for one electrode and graphite was used for the other electrode.

Note that a full cell refers to a cell of a lithium ion secondary battery including a positive electrode material, a negative electrode material, and an active material metal other than Li.

To see the difference depending on a nonaqueous solvent, Samples 1 to 7 with the same full-cell structure and different nonaqueous solvents were fabricated. Table 1 shows the components of positive electrodes, negative electrodes, and nonaqueous electrolytes used in this example.

TABLE 1

| | Positive electrode | | | Negative electrode | | | |
|---|---|---|---|---|---|---|---|
| | Active | Conductive | | Active | Thickening | | Nonaqueous |
| | material | additive | Binder | material | agent | Binder | electrolyte |
| Sample | LPF | GO | PVdF | Spherical | CMC | SBR | 1M |
| 1 | | | | natural | | | LiTFSA EMI-FSA |
| | | | | graphite | | | |
| Sample | LPF | GO | PVdF | Spherical | CMC | SBR | 1M |
| 2 | | | | natural | | | LiTFSA BMI-FSA |
| | | | | graphite | | | |
| Sample | LPF | GO | PVdF | Spherical | CMC | SBR | 1M |
| 3 | | | | natural | | | LiTFSA HMI-FSA |
| | | | | graphite | | | |
| Sample | LPF | GO | PVdF | Spherical | CMC | SBR | 1M |
| 4 | | | | natural | | | LiTFSA MOI-FSA |
| | | | | graphite | | | |
| Sample | LPF | GO | PVdF | Spherical | CMC | SBR | 1M |
| 5 | | | | natural | | | LiTFSA MNI-FSA |
| | | | | graphite | | | |
| Sample | LPF | GO | PVdF | Spherical | CMC | SBR | 1M |
| 6 | | | | natural | | | LiTFSA DMI-FSA |
| | | | | graphite | | | |
| Sample | LPF | GO | PVdF | Spherical | CMC | SBR | 1M |
| 7 | | | | natural | | | LiTFSA poEMI-FSA |
| | | | | graphite | | | |

The structural formulae of cations contained in the nonaqueous electrolytes are shown below.

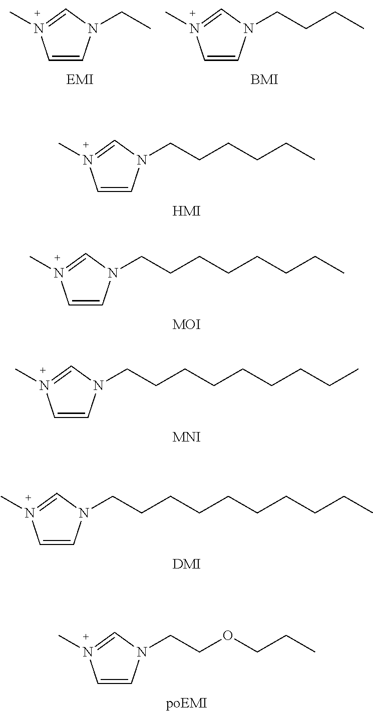

The nonaqueous electrolyte of Sample 1 was formed in such a manner that LiTFSA (abbreviation) which is an alkali metal salt was dissolved at a concentration of 1 mol/L in 1-ethyl-3-methylimidazolium bis(fluorosulfonyl)amide (abbreviation: EMI-FSA) which is an ionic liquid.

The nonaqueous electrolyte of Sample 2 was formed in such a manner that LiTFSA which is an alkali metal salt was dissolved at a concentration of 1 mol/L in 1-butyl-3-methylimidazolium bis(fluorosulfonyl)amide (abbreviation: BMI-FSA) which is an ionic liquid.

The nonaqueous electrolyte of Sample 3 was formed in such a manner that LiTFSA which is an alkali metal salt was dissolved at a concentration of 1 mol/L in 1-hexyl-3-methylimidazolium bis(fluorosulfonyl)amide (abbreviation: HMI-FSA) which is an ionic liquid.

The nonaqueous electrolyte of Sample 4 was formed in such a manner that LiTFSA which is an alkali metal salt was dissolved at a concentration of 1 mol/L in 3-methyl-1-octylimidazolium bis(fluorosulfonyl)amide (abbreviation: MOI-FSA) which is an ionic liquid.

The nonaqueous electrolyte of Sample 5 was formed in such a manner that LiTFSA which is an alkali metal salt was dissolved at a concentration of 1 mol/L in 3-methyl-1-nonylimidazolium bis(fluorosulfonyl)amide (abbreviation: MNI-FSA) which is an ionic liquid.

The nonaqueous electrolyte of Sample 6 was formed in such a manner that LiTFSA which is an alkali metal salt was dissolved at a concentration of 1 mol/L in 1-decyl-3-methylimidazolium bis(fluorosulfonyl)amide (abbreviation: DMI-FSA) which is an ionic liquid.

The nonaqueous electrolyte of Sample 7 was formed in such a manner that LiTFSA which is an alkali metal salt was dissolved at a concentration of 1 mol/L in 1-methyl-3-(2-propoxyethyl)imidazolium bis(fluorosulfonyl)amide (abbreviation: poEMI-FSA) which is an ionic liquid.

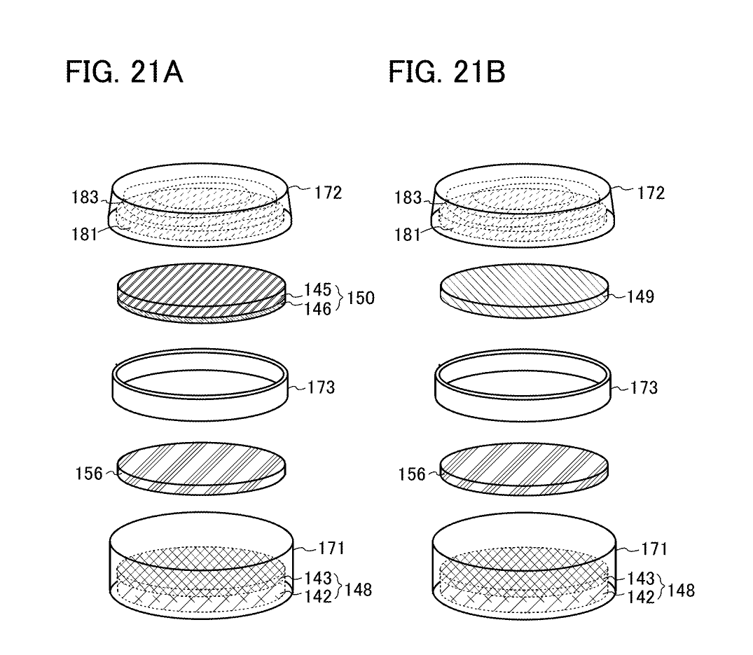

Here, fabrication methods of the samples in this example which are shown in Table 1 are each described with reference to FIG. 21A. Note that FIG. 21A illustrates the full-cell structure.

### (Fabrication Method of Full-Cell Structure of Samples 1 to 7)

Samples 1 to 7 each include a housing 171 and a housing 172 which serve as external terminals, a positive electrode 148, a negative electrode 150, a ring-shaped insulator 173, a separator 156, a spacer 181, and a washer 183.

The housing 171 and the housing 172 were formed of stainless steel (SUS). The spacer 181 and the washer 183 were also formed of stainless steel (SUS).

In the positive electrode 148, a positive electrode active material layer 143 containing a positive electrode active material, a conductive additive, and a binder in a weight ratio of 94.4:0.6:5 is provided over a positive electrode current collector 142 made of aluminum foil (15.958 φ). LiFePO₄ was used as the positive electrode active material. Graphene oxide (GO) was used as the conductive additive. Polyvinylidene fluoride (PVdF) was used as the binder. The positive electrode active material layer 143 had a thickness of 60 μm or larger and 70 μm or smaller and a density of 1.8 g/cc or higher and 2.0 g/cc or lower. The LiFePO₄ content per unit area of the positive electrode active material layer 143 was 7 mg/cm² or larger and 8 mg/cm² or smaller.

In the negative electrode 150, a negative electrode active material layer 146 containing a negative electrode active material, a first binder, and a second binder at a weight ratio of 97:1.5:1.5 is provided over a negative electrode current collector 145 made of aluminum foil (15.958φ). Note that spherical natural graphite was used as the negative electrode active material. Carboxymethyl cellulose (CMC) having a binding property was used as the first binder. Styrene-butadiene rubber (SBR) was used as the second binder. The negative electrode active material layer 146 had a thickness of 80 μm or larger and 90 μm or smaller and a density of 0.9 g/cc or higher and 1.2 g/cc or lower. The spherical natural graphite content per unit area of the negative electrode active material layer 146 was 8 mg/cm² or larger and 9 mg/cm² or lower.

For the separator 156, GF/C which is a glass fiber filter produced by Whatman Ltd. was used. The GF/C had a thickness of 260 μm.

In each of Samples 1 to 7, the positive electrode 148, the negative electrode 150, and the separator 156 were soaked in the nonaqueous electrolyte.

Then, as illustrated in FIG. 21A, the housing 171, the positive electrode 148, the separator 156, the ring-shaped insulator 173, the negative electrode 150, the spacer 181, the washer 183, and the housing 172 were stacked in this order with the housing 171 positioned at the bottom, and the housings 171 and 172 were crimped to each other with a “coin cell crimper”. Thus, Samples 1 to 7 were fabricated.

### (Evaluation of Initial Charge and Discharge Characteristics of Each Sample)

Next, the initial charge and discharge of each of Samples 1 to 7 were measured. The measurement of Samples 1 to 7 was performed with a charge-discharge measuring instrument (produced by TOYO SYSTEM Co., LTD) in a constant temperature bath at 60° C. In the measurement, constant current charge was performed at a rate of approximately 0.1 C (0.1 mA/cm²), and then discharge was performed at the same rate.

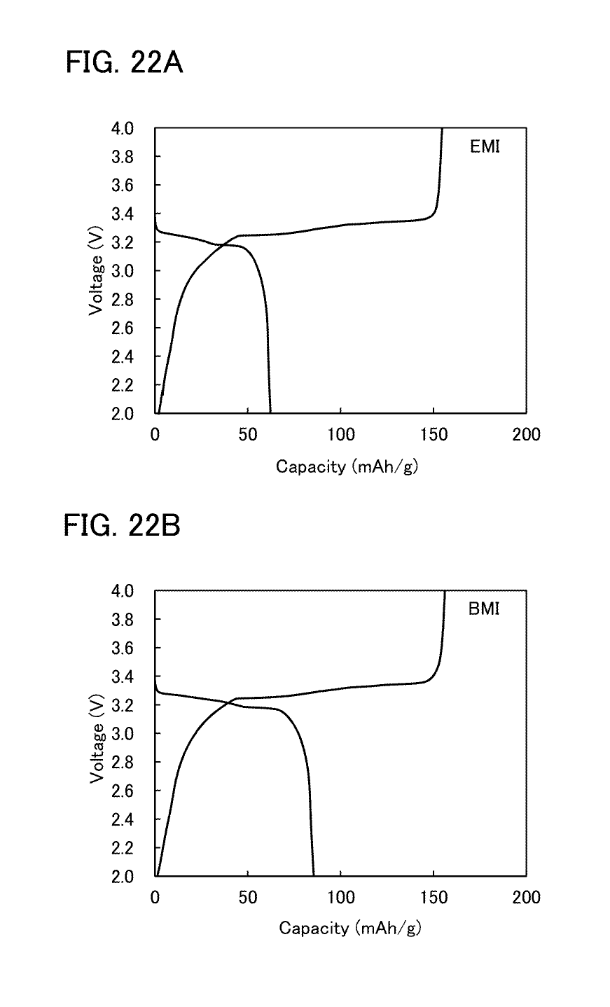

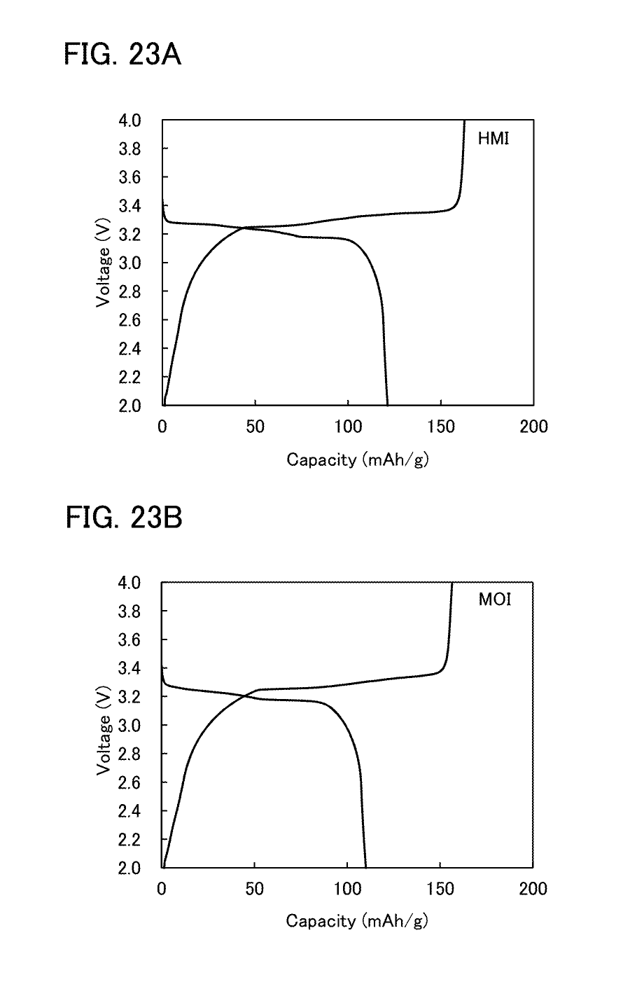

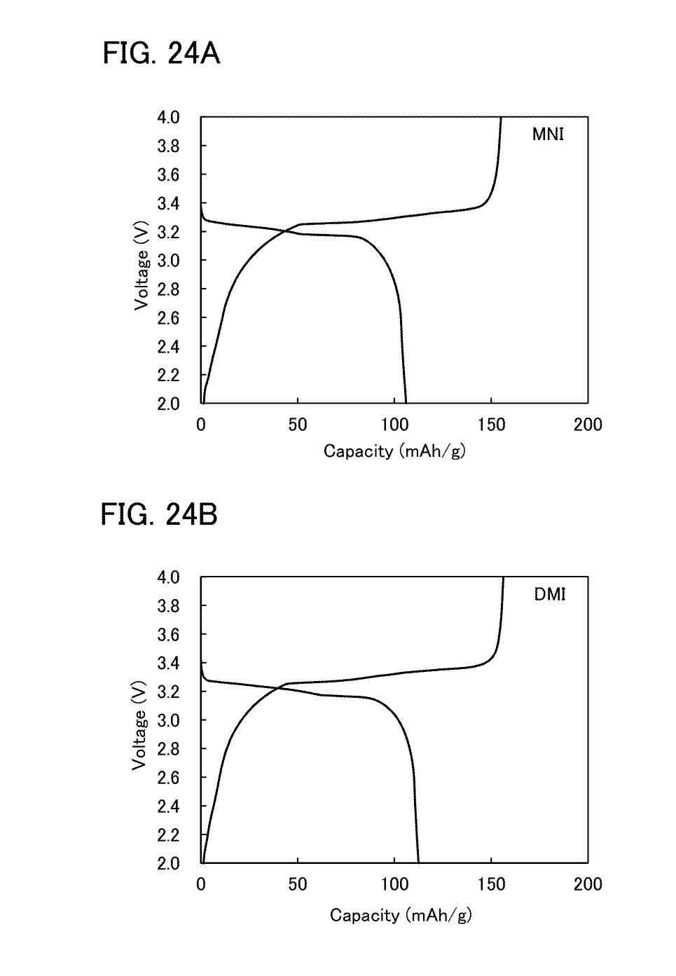

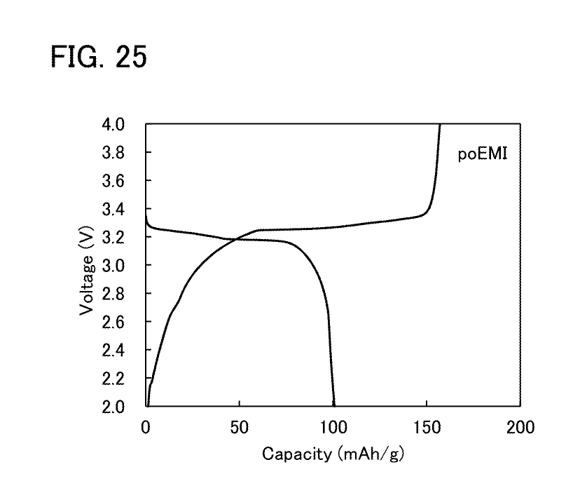

FIG. 22A, FIG. 22B, FIG. 23A, FIG. 23B, FIG. 24A, FIG. 24B, and FIG. 25 show the initial charge and discharge characteristics of Sample 1, Sample 2, Sample 3, Sample 4, Sample 5, Sample 6, and Sample 7, respectively. In each of FIGS. 22A and 22B, FIGS. 23A and 23B, and FIGS. 24A and 24B, and FIG. 25, the horizontal axis represents the capacity per weight (mAh/g) of the positive electrode active material and the vertical axis represents voltage (V).

As shown in FIGS. 22A and 22B, FIGS. 23A and 23B, FIGS. 24A and 24B, and FIG. 25, the discharge capacity at a cut-off voltage (2 V), which is a discharge characteristic, is 62 mAh/g in Sample 1, 86 mAh/g in Sample 2, 121 mAh/g in Sample 3, 110 mAh/g in Sample 4, 106 mAh/g in Sample 5, 113 mAh/g in Sample 6, and 101 mAh/g in Sample 7.

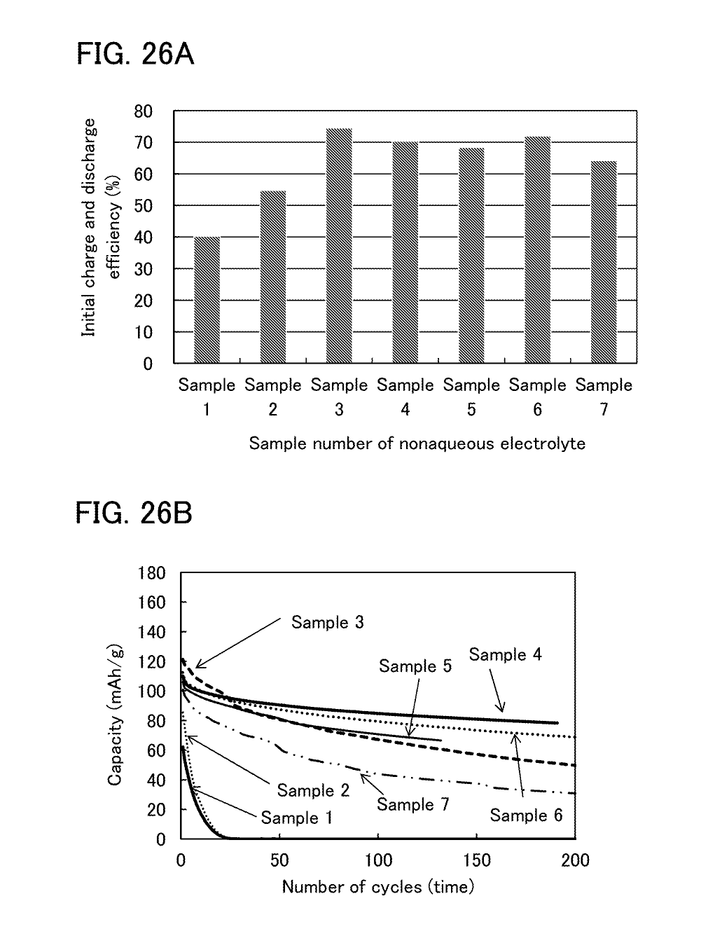

FIG. 26A shows the initial charge and discharge efficiency of each sample, and FIG. 26B shows the measurement results of the cycle characteristics thereof.

As shown in FIGS. 22A and 22B, FIGS. 23A and 23B, FIGS. 24A and 24B, and FIG. 25, favorable initial charge and discharge characteristics were obtained in Samples 2 to 7. In addition, as shown in FIG. 26A, the initial charge and discharge efficiency of each of Samples 2 to 7 is 50% or higher, which is favorable. Moreover, as shown in FIG. 26B, favorable cycle characteristics were obtained in Samples 3 to 7.

Consequently, according to the results of this example, it can be verified that Samples 2 to 7 in which an imidazolium cation has a straight chain formed of four or more atoms and including at least one of C and O have favorable battery characteristics.

### Example 7

In this example, power storage devices were fabricated with the use of the nonaqueous electrolyte of one embodiment of the present invention, and the power storage devices were evaluated. As the power storage devices, coin-type lithium ion secondary batteries were fabricated. The coin-type lithium ion secondary battery in this example has a lithium iron phosphate-lithium metal half-cell structure, in which lithium iron phosphate (LiFePO₄) was used for one electrode and lithium metal was used for the other electrode.

Note that a half cell refers to a cell of a lithium-ion secondary battery in which an active material other than a lithium metal is used for a positive electrode and a lithium metal is used for a negative electrode. In the half-cell structure described in this example, lithium iron phosphate was used as an active material of a positive electrode and a lithium metal was used as a negative electrode.

To see the difference depending on a nonaqueous solvent, Samples 8 and 9 with the same half-cell structure and different nonaqueous solvents were fabricated. Table 2 shows the components of positive electrodes, negative electrodes, and nonaqueous electrolytes used in this example.

TABLE 2

| | Positive electrode | | | | |
|---|---|---|---|---|---|
| | Active | Conductive | | Negative | Nonaqueous |
| | material | additive | Binder | electrode | electrolyte |
| Sample 8 | LPF | GO | PVdF | Li metal | 1M LiTFSA |
| | | | | | HMI-FSA |
| Sample 9 | LPF | GO | PVdF | Li metal | 1M LiTFSA |
| | | | | | poEMI-FSA |

The structural formulae of cations contained in the nonaqueous electrolytes are shown below.

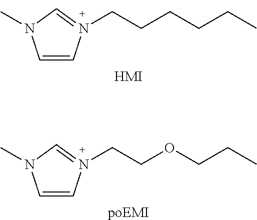

The nonaqueous electrolyte of Sample 8 was formed in such a manner that LiTFSA which is an alkali metal salt was dissolved at a concentration of 1 mol/L in 1-hexyl-3-methylimidazolium bis(fluorosulfonyl)amide (abbreviation: HMI-FSA) which is an ionic liquid.

The nonaqueous electrolyte of Sample 9 was formed in such a manner that LiTFSA which is an alkali metal salt was dissolved at a concentration of 1 mol/L in 1-methyl-3-(2-propoxyethyl)imidazolium bis(fluorosulfonyl)amide (abbreviation: poEMI-FSA) which is an ionic liquid.

Here, fabrication methods of the samples in this example which are shown in Table 2 are each described with reference to FIG. 21B. Note that FIG. 21B illustrates the half-cell structure.

### (Fabrication Method of Half-Cell Structure of Samples 8 and 9)

Samples 8 and 9 each include a housing 171 and a housing 172 which serve as external terminals, the positive electrode 148, a negative electrode 149, the ring-shaped insulator 173, the separator 156, the spacer 181, and the washer 183.

The housing 171 and the housing 172 were formed of stainless steel (SUS). The spacer 181 and the washer 183 were also formed of stainless steel (SUS).

In the positive electrode 148, a positive electrode active material layer 143 containing a positive electrode active material, a conductive additive, and a binder in a weight ratio of 94.4:0.6:5 is provided over a positive electrode current collector 142 made of aluminum foil (15.958 φ). LiFePO₄ was used as the positive electrode active material. Graphene oxide (GO) was used as the conductive additive. Polyvinylidene fluoride (PVdF) was used as the binder. The positive electrode active material layer 143 had a thickness of 60 μm or larger and 70 μm or smaller and a density of 1.8 g/cc or higher and 2.0 g/cc or lower. The LiFePO₄ content per unit area of the positive electrode active material layer 143 was 7 mg/cm² or larger and 8 mg/cm² or smaller.

A lithium metal was used as the negative electrode 149.

For the separator 156, GF/C which is a glass fiber filter produced by Whatman Ltd. was used. The GF/C had a thickness of 260 μm.

In each of Samples 8 and 9, the positive electrode 148, the negative electrode 149, and the separator 156 were soaked in the nonaqueous electrolyte.

Then, as illustrated in FIG. 21B, the housing 171, the positive electrode 148, the separator 156, the ring-shaped insulator 173, the negative electrode 149, the spacer 181, the washer 183, and the housing 172 were stacked in this order with the housing 171 positioned at the bottom, and the housings 171 and 172 were crimped to each other with a “coin cell crimper”. Thus, Samples 8 and 9 were fabricated.

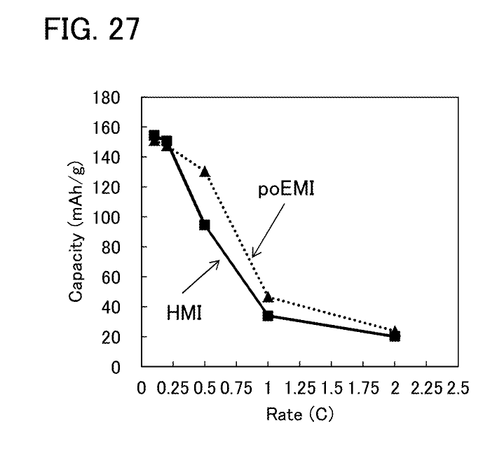

Next, the rate characteristics of Samples 8 and 9 were examined. The measurement was performed with a charge-discharge measuring instrument (produced by TOYO SYSTEM Co., LTD) in a constant temperature bath at 60° C. The charge voltage was lower than or equal to 4 V and the charge rate was 0.1 C, and the discharge rates were 0.1 C, 0.2 C, 0.5 C, 1 C, and 2 C. FIG. 27 shows discharge capacity versus rate. In FIG. 27, the horizontal axis represents discharge rate (C) and the vertical axis represents discharge capacity at 0.1 C. The results indicate that Sample 9 has better characteristics than Sample 8.

Accordingly, when a straight chain, which forms a substituent bonded to a nitrogen of the imidazolium cation, has the same number of atoms, the substituent preferably includes oxygen (O), in which case preferable battery characteristics can be obtained.

### Example 8

In this example, characteristics of the thin storage battery described in Embodiment 2, which is an example of a power storage device of one embodiment of the present invention, are described.

In a positive electrode, a positive electrode active material layer containing a positive electrode active material, a binder, and a conductive additive in a weight ratio of 94.4:0.6:5 is provided over a positive electrode current collector made of aluminum. LiFePO₄ was used as the positive electrode active material. Graphene oxide (GO) was used as the conductive additive. Polyvinylidene fluoride (PVdF) was used as the binder. The positive electrode active material layer had a thickness of 47 μm or larger and 53 μm or smaller and a density of 1.69 g/cc or higher and 2.06 g/cc or lower. The LiFePO₄ content per unit area of the positive electrode active material layer was 8.5 mg/cm² or larger and 9.1 mg/cm² or smaller.

In a negative electrode, a negative electrode active material layer containing a negative electrode active material, a first binder, and a second binder at a weight ratio of 97:1.5:1.5 is provided over a negative electrode current collector made of copper. Note that spherical natural graphite was used as the negative electrode active material. Carboxymethyl cellulose (CMC) having a binding property was used as the first binder. Styrene-butadiene rubber (SBR) was used as the second binder. The negative electrode active material layer had a thickness of 54 μm or larger and 58 μm or smaller and a density of 0.93 g/cc or higher and 1.07 g/cc or lower. The spherical natural graphite content per unit area of the negative electrode active material layer was 4.9 mg/cm² or larger and 5.7 mg/cm² or lower.

Next, thin storage batteries A to E were fabricated with the use of the positive electrode and the negative electrode. An aluminum film covered with a heat sealing resin was used as an exterior body. As a separator, 50-μm-thick solvent-spun regenerated cellulosic fiber (TF40, produced by NIPPON KODOSHI CORPORATION) was used.

One positive electrode and one negative electrode were used as electrodes of each thin storage battery and were arranged so that surfaces on which their respective active material layers were formed faced each other with the separator positioned therebetween.

The nonaqueous electrolyte of the thin storage battery A was formed in such a manner that LiTFSA (abbreviation) which is an alkali metal salt was dissolved at a concentration of 1 mol/L in 1-ethyl-3-methylimidazolium bis(fluorosulfonyl)amide (abbreviation: EMI-FSA) which is an ionic liquid.

The nonaqueous electrolyte of the thin storage battery B was formed in such a manner that LiTFSA which is an alkali metal salt was dissolved at a concentration of 1 mol/L in 3-methyl-1-propylimidazolium bis(fluorosulfonyl)amide (abbreviation: MPI-FSA) which is an ionic liquid.

The nonaqueous electrolyte of the thin storage battery C was formed in such a manner that LiTFSA which is an alkali metal salt was dissolved at a concentration of 1 mol/L in 1-butyl-3-methylimidazolium bis(fluorosulfonyl)amide (abbreviation: BMI-FSA) which is an ionic liquid.

The nonaqueous electrolyte of the thin storage battery D was formed in such a manner that LiTFSA which is an alkali metal salt was dissolved at a concentration of 1 mol/L in 1-hexyl-3-methylimidazolium bis(fluorosulfonyl)amide (abbreviation: HMI-FSA) which is an ionic liquid.

The nonaqueous electrolyte of the thin storage battery E was formed in such a manner that LiTFSA which is an alkali metal salt was dissolved at a concentration of 1 mol/L in 1-methyl-3-(2-propoxyethyl)imidazolium bis(fluorosulfonyl)amide (abbreviation: poEMI-FSA) which is an ionic liquid.

The viscosity of the ionic liquids and the nonaqueous electrolytes in the thin storage battery D and the thin storage battery E were measured. The viscosity of HMI-FSA was 48 mPa·s, and the viscosity of the nonaqueous electrolyte formed by dissolving LiTFSA at a concentration of 1 mol/L in HMI-FSA was 102 mPa·s. The viscosity of poEMI-FSA was 36.4 mPa·s, and the viscosity of the nonaqueous electrolyte formed by dissolving LiTFSA at a concentration of 1 mol/L in poEMI-FSA was 86.2 mPa·s.

In the thin storage battery D, the diffusion coefficient of a lithium ion in the nonaqueous electrolyte was 9.08×10⁻¹² m²/s at 25° C., 4.36×10⁻¹² m²/s at 10° C., 2.80×10⁻¹² m²/s at 0° C., 1.31×10⁻¹² m²/s at −10° C., and 3.27×10⁻¹³ m²/s at −25° C. In the thin storage battery E, the diffusion coefficient of a lithium ion in the nonaqueous electrolyte was 1.05×10⁻¹¹ m²/s at 25° C., 4.90×10⁻¹² m²/s at 10° C., 2.70×10⁻¹² m²/s at 0° C., 1.26×10⁻¹² m²/s at −10° C., and 3.07×10⁻¹³ m²/s at −25° C.

Next, the fabricated thin storage batteries A to E were subjected to aging. Note that for calculation of the rate, 1 C was set to 170 mA/g, which was the current value per weight of the positive electrode active material.

A flow of the aging is described. First, charging was performed at 25° C. at a rate of 0.01 C to an upper limit voltage of 3.2 V (Step 1).

Next, degasification was performed, and then, the batteries were sealed again (Step 2).

Subsequently, charging was performed at 25° C. at a rate of 0.05 C to an upper limit voltage of 4 V, and then, discharging was performed at a rate of 0.2 C to a lower limit voltage of 2 V (Step 3).

Then, charging and discharging were each performed twice alternately at 25° C. As the charging conditions, the upper limit voltage was set to 4 V and the rate was set to 0.2 C. As the discharging conditions, the lower limit voltage was set to 2 V and the rate was set to 0.2 C (Step 4).

Next, a charge-discharge cycle test of the fabricated thin storage batteries A to E was performed. The measurement temperature was 25° C. Here, the charge-discharge cycle test means repetition of cycles, where one cycle corresponds to one charging and one discharging after the charging. In the first cycle, charging and discharging were performed at a rate of 0.1 C. Subsequently, 200 cycles of charging and discharging were performed at a rate of 0.2 C, followed by one charge-discharge cycle at a rate of 0.1 C. After that, one charge-discharge cycle at a rate of 0.1 C was performed every 200 cycles at a rate of 0.2 C, and this procedure was repeated.

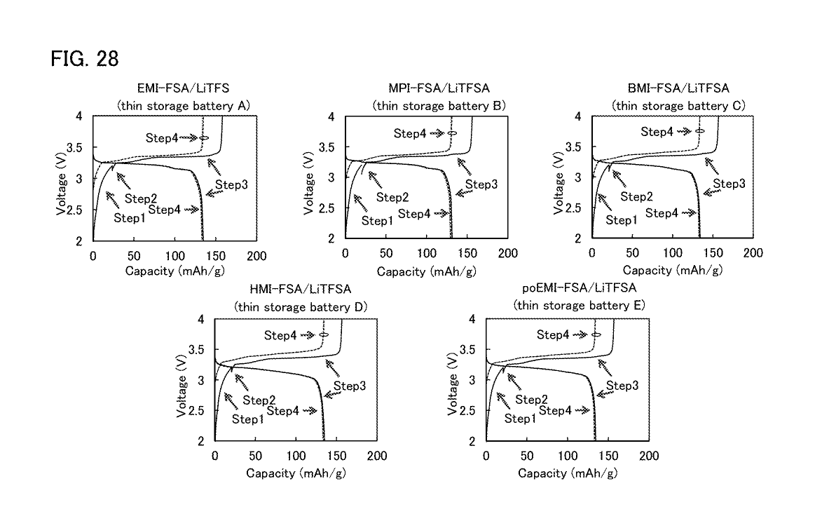

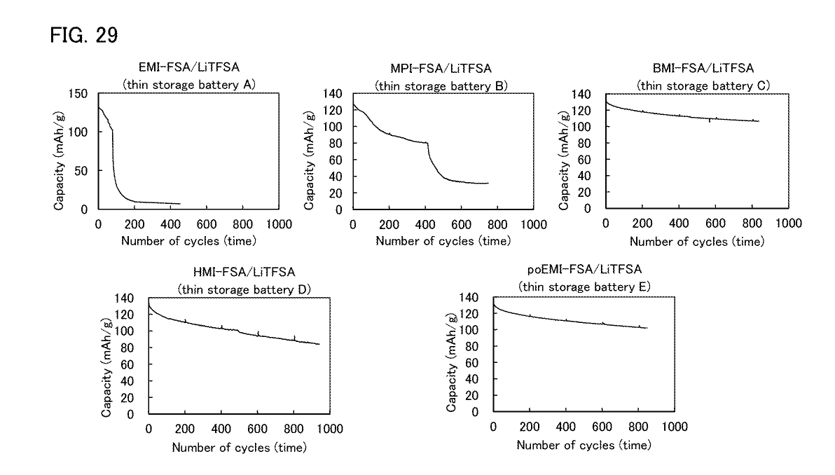

FIG. 28 shows the results of aging of the thin storage batteries A to E. FIG. 29 shows change in the discharge capacity with respect to the number of cycles of each of the thin storage batteries A to E.

As shown in FIG. 29, the thin storage batteries C to E have preferable cycle characteristics.

Consequently, according to the results of this example, it can be verified that the thin storage batteries C to E in which an imidazolium cation has a straight chain formed of four or more atoms and including at least one of C and O have favorable battery characteristics.

A thin storage battery D1 and a thin storage battery E1 were fabricated. Substances contained in negative electrodes of the thin storage batteries D1 and E1 are different from those in the thin storage batteries D and E.

In a negative electrode, a negative electrode active material layer containing a negative electrode active material, a first binder, a second binder, and a conductive additive at a weight ratio of 95:1.5:1.5:2 is provided over a negative electrode current collector made of copper. Note that spherical natural graphite was used as the negative electrode active material. Carboxymethyl cellulose (CMC) having a binding property was used as the first binder. Styrene-butadiene rubber (SBR) was used as the second binder. A vapor-grown carbon fiber (VGCF) was used as the conductive additive. The negative electrode active material layer had a thickness of 49 μm or larger and 55 μm or smaller and a density of 0.85 g/cc or higher and 0.92 g/cc or lower. The spherical natural graphite content per unit area of the negative electrode active material layer was 4.07 mg/cm² or larger and 4.42 mg/cm² or lower.

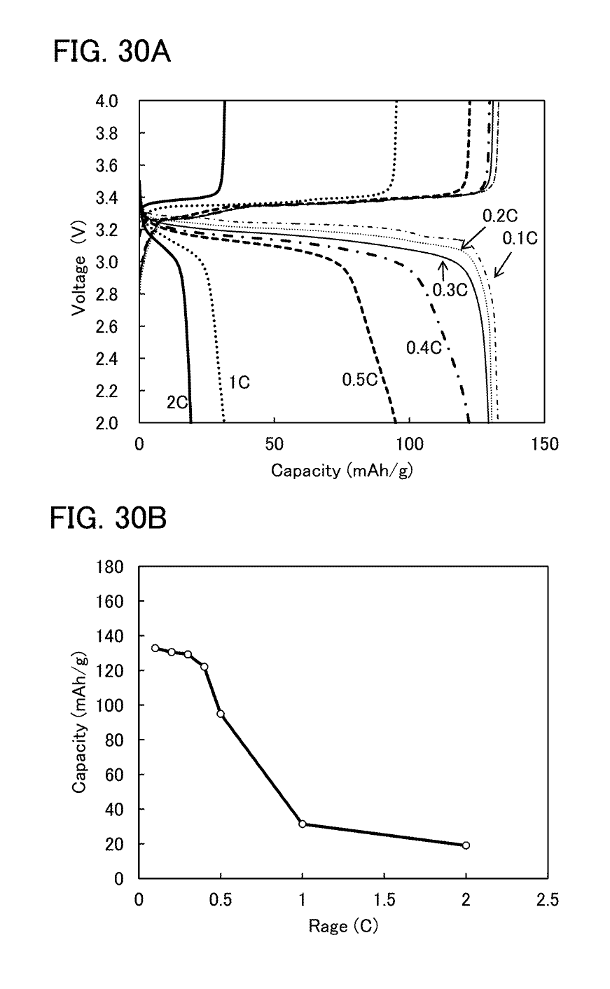

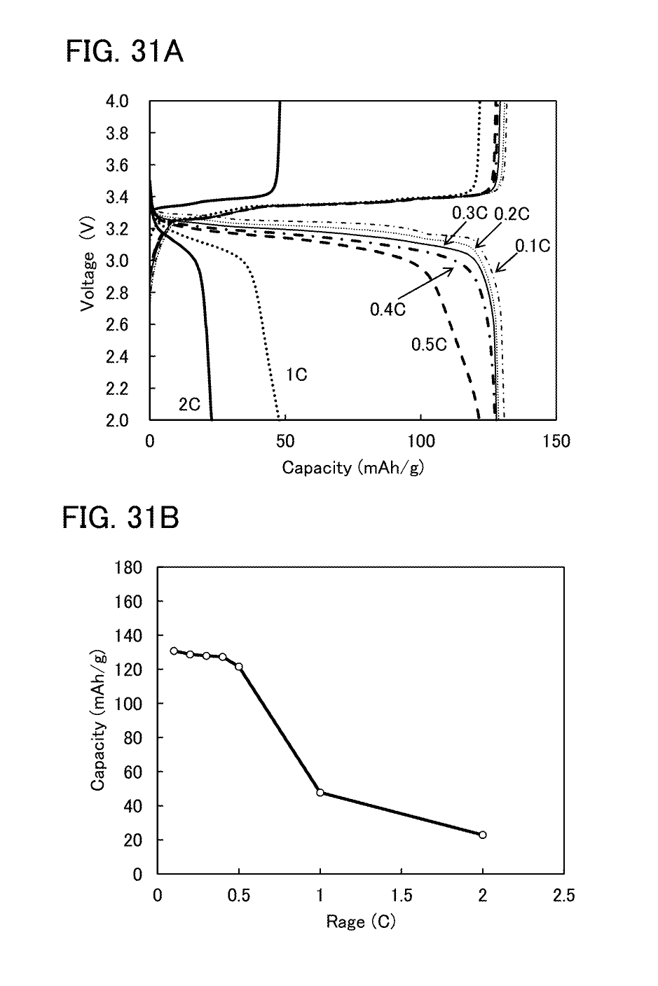

The rate characteristics of the fabricated thin storage batteries D1 and E1 were examined. The measurement was performed with a charge-discharge measuring instrument (produced by TOYO SYSTEM Co., LTD) in a constant temperature bath at 60° C. The charge voltage is lower than or equal to 4 V and the charge rate was 0.2 C, and discharge rates are 0.1 C, 0.2, 0.5 C, 1 C, and 2 C. FIG. 30A shows the charge and discharge characteristics of the thin storage battery D1 and FIG. 31A shows the charge and discharge characteristics of the thin storage battery E1. In FIG. 30A and FIG. 31A, the horizontal axis represents the capacity of the positive electrode active material per weight (mAh/g) and the vertical axis represents voltage (V). FIG. 30B shows the discharge capacity versus rate in the thin storage battery D1 and FIG. 31B shows the discharge capacity versus rate in the thin storage battery E1. In FIG. 30B and FIG. 31B, the horizontal axis represents discharge rate (C) and the vertical axis represents discharge capacity at 0.1 C. These results indicate that the thin storage battery E1 has better characteristics than the thin storage battery D1.

The temperature dependence of charge and discharge characteristics of the thin storage battery D1 and the thin storage battery E1 were examined. The measurement was performed with a charge-discharge measuring instrument (produced by TOYO SYSTEM Co., LTD) in a constant temperature bath. The measurement temperatures were 25° C., 10° C., 0° C., −10° C., and −25° C. In the measurement, constant current charge was performed at a rate of 0.1 C, and then discharge was performed at a rate of 0.2 C. Note that the charge was performed at 25° C.

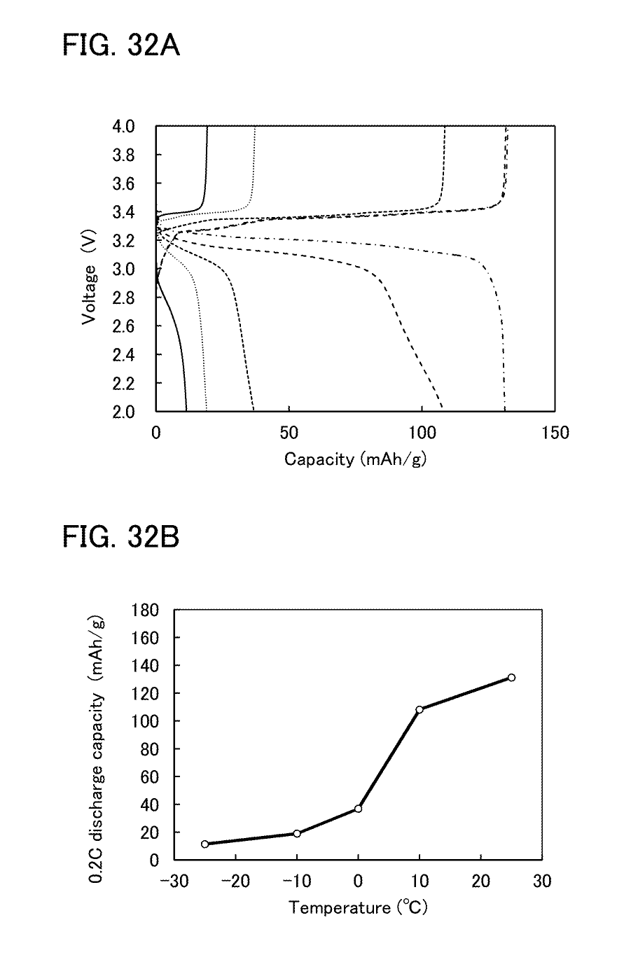

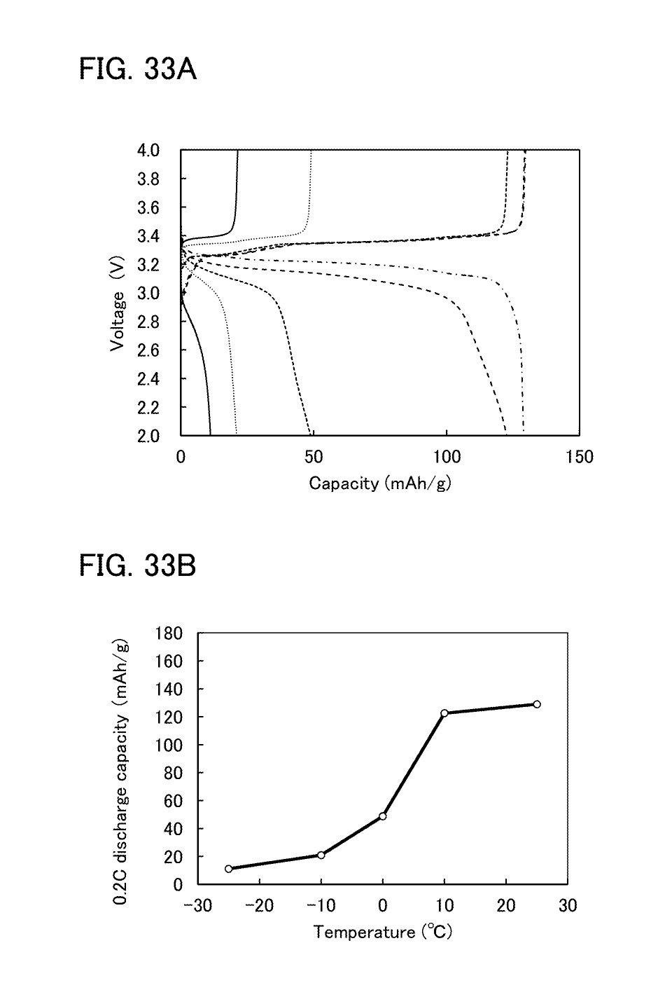

FIGS. 32A and 32B and FIGS. 33A and 33B show the measurement results. FIG. 32A shows the charge and discharge characteristics of the thin storage battery D1 and FIG. 33A shows the charge and discharge characteristics of the thin storage battery E1. Each of FIG. 32A and FIG. 33A shows the results at −25° C., −10° C., 0° C., 10° C., and 25° C. in this order from the left side. FIG. 32B shows the relation between the temperature and the discharge capacity at 0.2 C in the thin storage battery D1. FIG. 33B shows the relation between the temperature and the discharge capacity at 0.2 C in the thin storage battery E1. These results indicate that the thin storage battery E1 has better characteristics than the thin storage battery D1 at a low temperature lower of 0° C. or lower.

Accordingly, when a straight chain, which forms a substituent bonded to a nitrogen of the imidazolium cation, has the same number of atoms, the substituent preferably includes oxygen (O), in which case preferable battery characteristics can be obtained.

### Example 9

In this example, description is given on differential scanning calorimetry (DSC) of HMI-FSA (abbreviation) and poEMI-FSA (abbreviation) which are nonaqueous solvents included in nonaqueous electrolytes of embodiments of the present invention.

The samples were each cooled by decreasing a temperature from room temperature to around −120° C. at a rate of −10° C./min in an air atmosphere, and then heated by increasing the temperature from around −120° C. to 100° C. at a rate of 10° C./min. Then, the samples were each cooled by decreasing the temperature from 100° C. to −100° C. at a rate of −10° C./min, heated by increasing the temperature from −100° C. to 100° C. at a rate of 10° C./min, and cooled by decreasing the temperature to −120° C. at a rate of −10° C./min. Then, the calorimetry was performed while the samples were heated by increasing the temperature from −100° C. to 100° C. at a rate of 10° C./min.

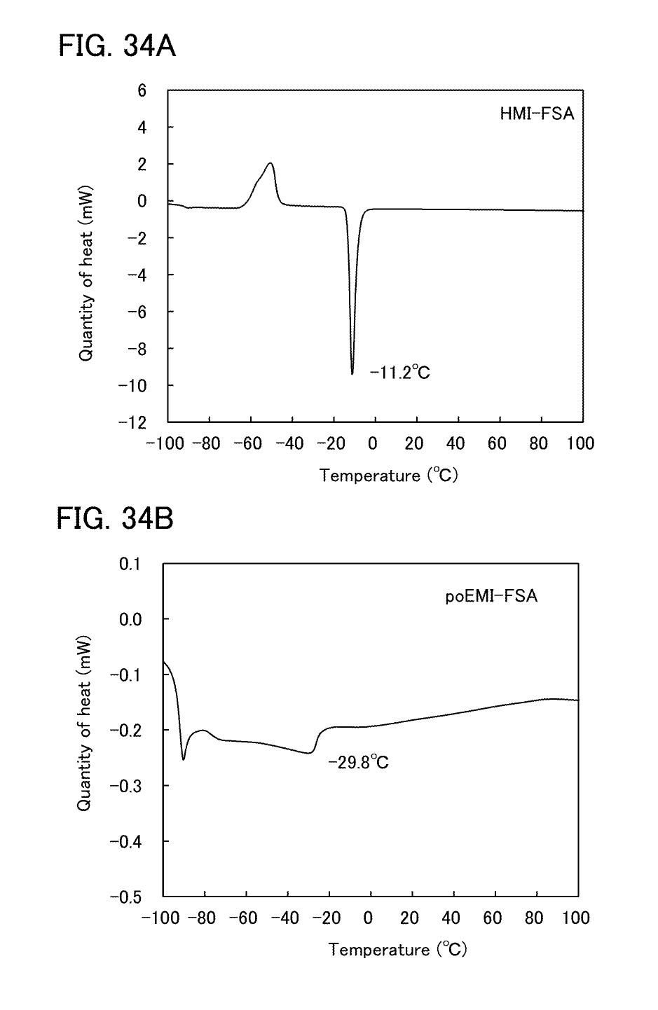

FIG. 34A shows the DSC measurement results of HMI-FSA and FIG. 34B shows the DSC measurement results of poEMI-FSA. In FIGS. 34A and 34B, the vertical axis represents quantity of heat [mW] and the horizontal axis represents temperature [° C.].

FIGS. 34A and 34B indicate that HMI-FSA has a melting point of approximately −11.2° C. and poEMI-FSA has a melting point of approximately −29.8° C.

Accordingly, when a straight chain, which forms a substituent bonded to a nitrogen of the imidazolium cation, has the same number of atoms, the substituent preferably includes oxygen (O), in which case preferable battery characteristics can be obtained.

This application is based on Japanese Patent Application serial No. 2013-237147 filed with Japan Patent Office on Nov. 15, 2013, Japanese Patent Application serial No. 2013-237158 filed with Japan Patent Office on Nov. 15, 2013, and Japanese Patent Application serial No. 2014-149489 filed with Japan Patent Office on Jul. 23, 2014, the entire contents of which are hereby incorporated by reference.

What is claimed is:

1. A compound comprising:
a cation represented by formula (G1): 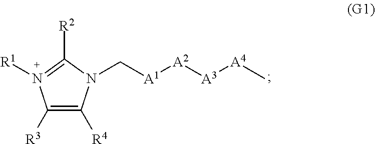
and
an anion,
wherein:
R¹ represents an alkyl group having 1 to 4 carbon atoms;
R² to R⁴ each independently represent a hydrogen atom or an alkyl group having 1 to 4 carbon atoms;
A¹ to A⁴ each independently represent a methylene group or an oxygen atom; and
at least one of A¹ to A⁴ represents an oxygen atom.

2. The compound according to claim 1, wherein the cation is represented by formula (G2):

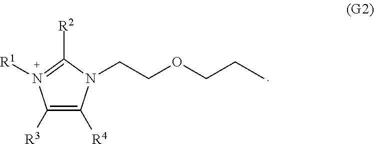

3. The compound according to claim 1, wherein the cation is represented by formula (G3):

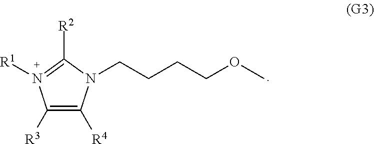

4. The compound according to claim 1, wherein the anion is any one of a monovalent amide anion, a monovalent methide anion, a fluorosulfonate anion (SO₃F⁻), a perfluoroalkylsulfonate anion, a tetrafluoroborate anion (BF₄⁻), a perfluoroalkylborate anion, a hexafluorophosphate anion (PF₆⁻), and a perfluoroalkylphosphate anion.

5. The compound according to claim 1, wherein the anion is a bis(fluorosulfonyl)amide anion.

6. The compound according to claim 1, wherein the alkyl group represented by R² to R⁴ is a methyl group.

7. The compound according to claim 1, wherein the cation is represented by any one of formulae (102), (104), (106), and (107):

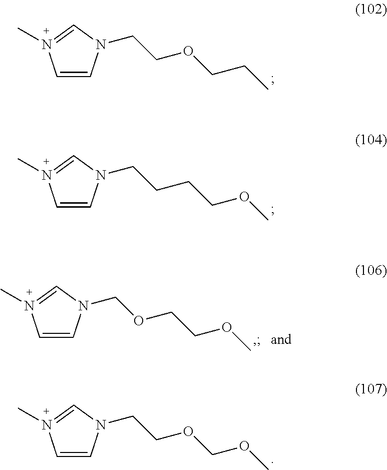

8. A nonaqueous electrolyte comprising an alkali metal salt and the compound according to claim 1.

9. A power storage device comprising the nonaqueous electrolyte according to claim 8.

10. A device comprising:
a cation and an anion,
wherein:
the cation has a five-membered heteroaromatic ring having one or more substituents; and
at least one of the substituents is a straight chain formed of four or more atoms and includes one or more of carbon, oxygen, silicon, nitrogen, sulfur, and phosphorus.

11. The device according to claim 10, wherein the five-membered heteroaromatic ring is a monocyclic five-membered heteroaromatic ring.

12. The device according to claim 11, wherein the cation is an imidazolium cation.

13. The device according to claim 10, wherein at least one heteroatom in the five-membered heteroaromatic ring has the straight chain.

14. The device according to claim 10, wherein the five-membered heteroaromatic ring includes at least one nitrogen atom.

15. The device according to claim 10, wherein the anion is any one of a monovalent amide anion, a monovalent methide anion, a fluorosulfonate anion (SO₃F⁻), a perfluoroalkylsulfonate anion, a tetrafluoroborate anion (BF₄⁻), a perfluoroalkylborate anion, a hexafluorophosphate anion (PF₆⁻), and a perfluoroalkylphosphate anion.

16. The device according to claim 10, further comprising an alkali metal salt.

17. The device according to claim 16, wherein the alkali metal salt is a lithium salt.

18. The device according to claim 10, wherein the device is a power storage device.
